(19) United States
(12) Patent Application Publication
(10) Pub. No.: US 2025/0104240 A1
(43) Pub. Date: Mar. 27, 2025

(54) PERSONALIZED PROFILING OF FUTURE BRAIN TRAJECTORIES AND FUTURE DISEASE EVOLUTION USING GENERATIVE ARTIFICIAL INTELLIGENCE

(71) Applicant: UNIVERSITY OF SOUTHERN CALIFORNIA, Los Angeles, CA (US)

(72) Inventor: Andrei Irimia, Los Angeles, CA (US)

(73) Assignee: UNIVERSITY OF SOUTHERN CALIFORNIA, Los Angeles, CA (US)

(21) Appl. No.: 18/887,555

(22) Filed: Sep. 17, 2024

Related U.S. Application Data
(60) Provisional application No. 63/540,475, filed on Sep. 26, 2023.

(51) Int. Cl. G06T 7/00; A61B 5/00; A61B 5/055; G16H 30/40

(52) U.S. Cl. CPC G06T 7/0014; A61B 5/0042; A61B 5/055; A61B 5/4064; G16H 30/40; G06T 2207/10088; G06T 2207/20076; G06T 2207/20081; G06T 2207/20084; G06T 2207/30016

(57) ABSTRACT

A system, method, and device (“system”) is provided for conducting anatomically interpretable deep learning of brain age that captures domain-specific cognitive impairment. By performing personalized profiling of future brain trajectories using generative artificial intelligence, the system facilitates early identification of neuroanatomy changes to screen individuals according to risk of neurocognitive impairments. A system may implement a generative AI model and may receive a plurality of brain data sets, extract various maps of a subject brain, and determine a salience probability map of future brain trajectory or future brain disease biomarkers based thereon. Moreover, the generative AI model may compute a training objective and the model may update based on the training objective.

### CROSS REFERENCE TO RELATED APPLICATIONS

This application is based upon and claims the benefit of and priority to U.S. Provisional Patent Application No. 63/540,475 entitled “PERSONALIZED PROFILING OF FUTURE BRAIN TRAJECTORIES AND FUTURE DISEASE EVOLUTION USING GENERATIVE ARTIFICIAL INTELLIGENCE,” filed on Sep. 26, 2023, the entire content of which is incorporated by reference herein.

### STATEMENT REGARDING GOVERNMENT RIGHTS

This invention was made with government support under grant no. R01 AG 079957, awarded by the (NIH) National Institutes of Health. The government has certain rights in the invention.

### BACKGROUND

### 1. Field

The following disclosure relates to processing of medical imaging, and more specifically, to personalized profiling of future brain trajectories and future disease evolution using generative artificial intelligences to process medical imaging.

### 2. Description of the Related Art

The gap between chronological age (CA) and biological brain age, as estimated from magnetic resonance images (MRIs), reflects how individual patterns of neuroanatomic aging deviate from their typical trajectories. MRI-derived brain age (BA) estimates are often obtained using deep learning models that may perform relatively poorly on new data or that lack neuroanatomic interpretability. Early identification of neuroanatomy changes can help to screen individuals according to their Alzheimer's disease (AD) risk. The phenotypic age of the human brain, as revealed via deep learning of anatomic magnetic resonance images, reflects patterns of structural change related to cognitive decline. This disclosure provides interpretable deep learning estimates that the brain ages that are more accurate than any other approaches to date. Furthermore, compared to chronological age, this system provides inferred brain ages significantly more strongly associated with early signs of Alzheimer's disease. Maps conveying the importance of each brain region for estimating brain age reveal differences in patterns of neurological aging between males and females and between persons with and without cognitive impairment. These findings provide insight into early identification of persons at high risk of Alzheimer's disease.

### SUMMARY

A method is provided. The method may include computing, via a generative artificial intelligence (“AI”) module, a training objective for a generative artificial intelligence (“AI”) model based on a training data set of a plurality of cognitively normal brain data sets. Each cognitively normal brain data set in the plurality of cognitively normal brain data sets may include cognitively normal multi-dimensional brain imaging data corresponding to a cognitively normal brain of a cognitively normal participant to form the generative AI model. The method may include receiving, via the generative AI module, a plurality of brain data sets, each of the plurality of brain data sets including subject multi-dimensional brain imaging data, each of the plurality of brain data sets corresponding to a subject brain of each of a plurality of subjects. The method may include extracting, via the generative AI module and from the subject multi-dimensional brain imaging data, a saliency map of future brain trajectory or future brain disease biomarkers for the subject brain of each of the plurality of subjects. The method may include determining, via the generative AI module, a saliency probability map of future brain trajectory or future brain disease biomarkers for each of the plurality of subjects based on the saliency map of future brain trajectory or future brain disease biomarkers and the generative AI model. The method may include estimating, via the generative AI module and through the generative AI model, one of a future brain trajectory or future brain disease biomarkers for the subject brain of each of the plurality of subjects based on the saliency map of future brain trajectory or future brain disease biomarkers. The method may include updating, via the generative AI module, the generative AI model based on the training objective.

In various embodiments, the method may include one or more additional feature. A few additional features are provided in this and the following paragraphs. For instance, the training objective of the generative AI model may include a generative neural network or an ensemble of generative, autoencoder, or diffusion neural networks. The subject multi-dimensional brain imaging data may be a T1-weighted, T2-weighted, diffusion-weighted, fMRI-weighted, or FLAIR-weighted magnetic resonance image.

Determining the saliency probability map of future brain trajectory, including a brain aging trajectory and disease biomarker trajectory, for the subject brain of each of the plurality of subjects may include calculating the saliency probability map of future brain trajectory or future brain disease biomarkers based on a sex of each of the plurality of subjects. Determining the saliency probability map of future brain trajectory, including the brain aging trajectory and disease biomarker trajectory, for the subject brain of each of the plurality of subjects may include calculating an average of the saliency probability map of future brain trajectory or future brain disease biomarkers based on a deviation from the training data set.

The saliency probability map of future brain trajectory, including a brain aging trajectory and disease biomarker trajectory, may be determined by dividing the saliency probability map of future brain trajectory or future brain disease biomarkers at each of a plurality of brain locations by a sum of all brain saliencies. The cognitively normal multi-dimensional brain imaging data may be generated by pre-processing a magnetic resonance image for skull-stripping and registering of the cognitively normal brain of the cognitively normal participant into a common coordinate space.

In various embodiments, the method also includes further analyzing aspects. For instance the method may include analyzing, at a surface level, the saliency probability map of future brain trajectory, including brain aging trajectory and disease biomarker trajectory, for the subject brain of each of the plurality of subjects. The method may include analyzing, at a volume level, the saliency probability map of future brain trajectory, including the brain aging trajectory and disease biomarker trajectory, for the subject brain of each of the plurality of subjects. In various embodiments, and in response to the analyzing at the surface level and the analyzing at the volume level, confounding effects of differences in brain shape and size are removed.

In various embodiments, at the surface level, saliencies from the saliency probability map of future brain trajectory, including the brain aging trajectory and disease biomarker trajectory, are projected to a native cortical surface. In various embodiments, at the surface level, saliencies from the saliency probability map of future brain trajectory, including the brain aging trajectory and disease biomarker trajectory, are projected onto a cortical mantle as a cortical overlay by volume to surface mapping. In various embodiments, at the surface level, a mean value for the saliency map of future brain trajectory, including the brain aging trajectory and disease biomarker trajectory, is calculated at a surface vertex by averaging the saliency map of future brain trajectory across cortical ribbon voxels within a cylinder according to a Gaussian weighted function. In various embodiments, the saliency probability map of future brain trajectory, including the brain aging trajectory and disease biomarker trajectory, determined for the subject brain of each of the plurality of subjects is calculated by dividing the saliency probability map of future brain trajectory or future brain disease biomarkers at each of a plurality of brain locations by a sum of all brain saliencies at both the surface level and the volume level.

In various embodiments, the method includes transmitting, via the generative AI module, the saliency probability map of future brain trajectory, including brain aging trajectory and disease biomarker trajectory, for the subject brain of each of the plurality of subjects to a display device. The saliency probability map of future brain trajectory, including the brain aging trajectory and disease biomarker trajectory, for the subject brain of each of the plurality of subjects may be displayed on the display device in response to the transmitting.

A method is provided. The method may be for estimating a saliency probability map of future brain trajectory, including brain aging trajectory and disease biomarker trajectory, of a patient. The method may include pre-processing a magnetic resonance image (MRI) of the patient to reconstruct and segment the MRI to form a T1-weighted, T2-weighted, diffusion-weighted, fMRI-weighted, or FLAIR-weighted MRI. The method may include inputting the T1-weighted, T2-weighted, diffusion-weighted, fMRI-weighted, or FLAIR-weighted MRI into a generative artificial intelligence (“AI”) module. The generative AI module may be configured to compute a training objective for a generative artificial intelligence (“AI”) model based on a training data set of a plurality of cognitively normal brain data sets. Each cognitively normal brain data set in the plurality of cognitively normal brain data sets may include cognitively normal multi-dimensional brain imaging data corresponding to a cognitively normal brain of a cognitively normal participant. The method may include receiving, via the generative AI module, an estimated saliency map of the patient for future brain trajectory or future brain disease biomarkers. The method may include updating, via the generative AI module, the generative AI model based on the training objective.

One or more further aspects may also be provided. For instance, in various embodiments of the method in response to the inputting the T1-weighted, T2-weighted, diffusion-weighted, fMRI-weighted, or FLAIR-weighted MRI into the generative AI module, the generative AI module determines a saliency map of future brain trajectory or future brain disease biomarkers based on the T1-weighted, T2-weighted, diffusion-weighted, fMRI-weighted, or FLAIR-weighted MRI. In various embodiments, determining the saliency map of future brain trajectory or future brain disease biomarkers includes calculating an average saliency probability map of future brain trajectory or future brain disease biomarkers based on a sex of the patient.

An article of manufacture is also provided. The article may include a tangible, non-transitory computer-readable storage medium having instructions stored thereon that, in response to execution by one or more processors, cause the one or more processors to perform operations. The operations may include receiving, via the one or more processors, a plurality of brain data sets. Each of the plurality of brain data sets may include subject multi-dimensional brain imaging data. Each of the plurality of brain data sets may correspond to a subject brain of each of a plurality of subjects. The operations may include extracting, via the one or more processors, a cognitive or affective saliency map for the subject brain of each of the plurality of subjects. The operations may include determining, via the one or more processors, a saliency probability map of future brain trajectory or future brain disease biomarkers for the subject brain of each of the plurality of subjects based on a saliency map of future brain trajectory or future brain disease biomarkers and a generative artificial intelligence (“AI”) model. The generative AI model may be configured to compute a training objective based on a training data set of a plurality of cognitively normal brain data sets. Each cognitively normal brain data set in the plurality of cognitively normal brain data sets may include cognitively normal multi-dimensional brain imaging data corresponding to a cognitively normal brain of a cognitively normal participant. The method may include updating, via the one or more processors, the generative AI model based on the training objective.

In various embodiments of the article of manufacture, the determining the saliency probability map of future brain trajectory may also include brain aging trajectory and disease biomarker trajectory, for the subject brain of each of the plurality of subjects and includes calculating an average saliency probability map for each sex and a cognitive status.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the following illustrative figures. In the following figures, like reference numbers refer to similar elements and steps throughout the figures.

FIG. 1 illustrates an example system, in accordance with various embodiments;

FIG. 2 illustrates a flow chart of a process capable of being performed by the example system, in accordance with various embodiments;

FIG. 3 illustrates a method of estimating a saliency probability map of future brain trajectory, in accordance with various embodiments;

FIG. 4A illustrates an example chart of aggregate datasets associated with the Alzheimer's Disease Neuroimaging Initiative (ADNI), in accordance with various embodiments;

FIG. 4B shows T1-Weighted MRIs, in accordance with various embodiments;

FIG. 4C shows skull-stripped MRIs, in accordance with various embodiments;

FIG. 4D shows saliency maps, in accordance with various embodiments;

FIG. 4E shows average saliency maps, in accordance with various embodiments;

FIG. 4F shows aspects of a processing mechanism, in accordance with various embodiments;

FIG. 4G shows a chart illustrating data for male and female participants, in accordance with various embodiments;

FIG. 4H shows a chart of neural and cognitive measures, in accordance with various embodiments;

FIG. 5A illustrates sex-specific mean saliency maps and a sex dimorphism map of participants, in accordance with various embodiments;

FIG. 5B shows an illustration of canonical views of a sex dimorphism map for participants, in accordance with various embodiments;

FIG. 5C illustrates mean saliency maps and a dimorphism map of participants comparing CI and CN participants, in accordance with various embodiments;

FIG. 5D shows an illustration of canonical views of a dimorphism map for participants comparing CI and CN participants, in accordance with various embodiments;

FIG. 6A illustrates a chart showing that older BA and CA are correlated with worse performance on various tests;

FIG. 6B shows a chart illustrating that no neurocognitive measure examined is significantly more correlated with BA than with CA;

FIG. 6C shows a chart illustrating correlation of BA and CA with scores on measures of neurocognitive function, in accordance with various embodiments;

FIG. 6D shows a chart illustrating neurocognitive measures for participants with any type of CI, in accordance with various embodiments;

FIG. 7 illustrates a chart of radar plots of sex-specific MAEs and performance parameters, in accordance with various embodiments; and

FIG. 8 depicts a table of data according to various participant demographics, in accordance with various embodiments.

### DETAILED DESCRIPTION

A system, apparatus and/or method for personalized profiling of future brain trajectories using generative artificial intelligence is disclosed herein.

The gap between chronological age (CA) and biological brain age, as estimated from magnetic resonance images (MRIs), reflects how individual patterns of neuroanatomic aging deviate from their typical trajectories. MRI-derived brain age (BA) estimates are often obtained using deep learning models that may perform relatively poorly on new data or that lack neuroanatomic interpretability. This study introduces a convolutional neural network (CNN) to estimate BA after training on the MRIs of 4,681 cognitively normal (CN) participants and testing on 1,170 CN participants from an independent sample. BA estimation errors are notably lower than those of previous studies. At both individual and cohort levels, the CNN provides detailed anatomic maps of brain aging patterns that reveal sex dimorphisms and neurocognitive trajectories in adults with mild cognitive impairment (MCI, N=351) and Alzheimer's disease (AD, N=359). In individuals with MCI (54% of whom were diagnosed with dementia within 10.9 y from MRI acquisition), BA is significantly better than CA in capturing dementia symptom severity, functional disability, and executive function. Profiles of sex dimorphism and lateralization in brain aging also map onto patterns of neuroanatomic change that reflect cognitive decline. Significant associations between BA and neurocognitive measures suggest that the proposed framework can map, systematically, the relationship between aging-related neuroanatomy changes in CN individuals and in participants with MCI or AD. Early identification of such neuroanatomy changes can help to screen individuals according to their AD risk.

Although chronological age (CA) reflects disease risk, the rate of aging varies across individuals, organs, tissues, and clinical conditions. Because CA does not capture this variation well, there is interest in estimating biological age to predict morbidity. Among typically aging adults, in the absence of any clinical indications, biological age is expected to equal CA, on average. Neuroanatomic biological age inferred from MRI, henceforth referred to as brain age (BA), can quantify disease-related changes in aging and associated increases in mortality risk. Thus, reliable BA estimators can help to stratify individuals according to disease risk. The difference between BA and CA, known as age gap (AG), conveys whether aging is faster or slower than expected. In clinical cohorts, improving BA estimates can translate into better estimates of participants' deviations from typical aging. For example, BA has the potential to become an affordable and noninvasive preclinical indicator of mild cognitive impairment (MCI) and Alzheimer's disease (AD) due to the strong association between BA and dementia risk.

Deep learning (DL) methods can estimate BA by learning to estimate cognitively normal (CN) subjects' CAs from MRIs of their brain, while minimizing the mean absolute error (MAE) between BA and CA. Compared to other approaches, DL typically yields better BA estimates. However, its inherent black-box nature hinders the interpretability of its feature attribution, since the relative utility of regional brain features for BA estimation by DL methods is unknown. Furthermore, many DL estimators of BA are inaccurate and lack generalizability to cohorts not encountered during DL training. To address these shortcomings, this disclosure introduces an interpretable three-dimensional (3D) convolutional neural network (CNN) to estimate BA from T₁-weighted brain MRIs. To provide neuroanatomic interpretability, MRI feature attribution is achieved through saliency maps. These allow one to identify structural brain patterns of CN aging that reflect regional and sex-specific variations in neuroanatomic features reflecting BA. 3D-CNN generalizability to new cohorts is also illustrated. The translational potential of this study is reflected in the associations between estimated BAs and neurocognitive measures of CI.

One significance of this disclosure is described in the following paragraph. Specifically, the phenotypic age of the human brain, as revealed via deep learning of anatomic magnetic resonance images, reflects patterns of structural change related to cognitive decline. The interpretable deep learning estimates of this disclosure estimates brain ages more accurately than any other approaches to date. Furthermore, compared to chronological age, the inferred brain ages are significantly more strongly associated with early signs of Alzheimer's disease. Maps conveying the importance of each brain region for estimating brain age reveal differences in patterns of neurological aging between males and females and between persons with and without cognitive impairment. These findings provide insight into early identification of persons at high risk of Alzheimer's disease.

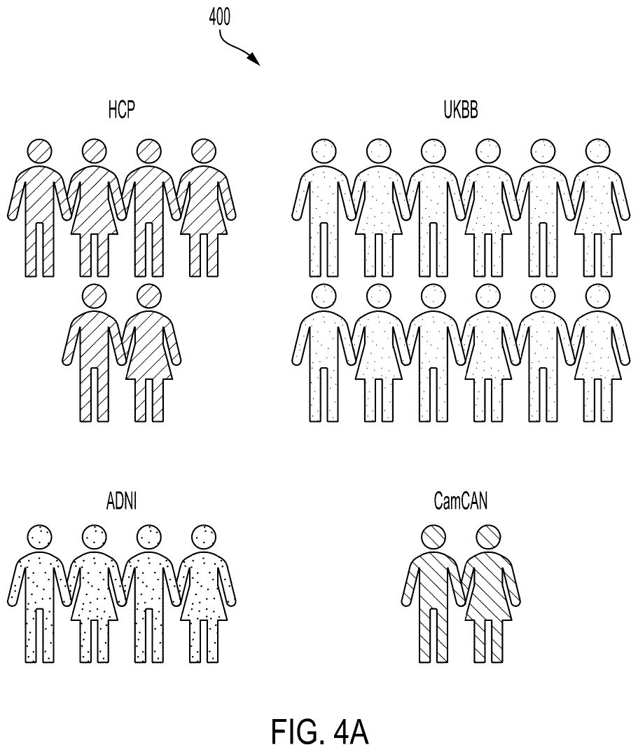

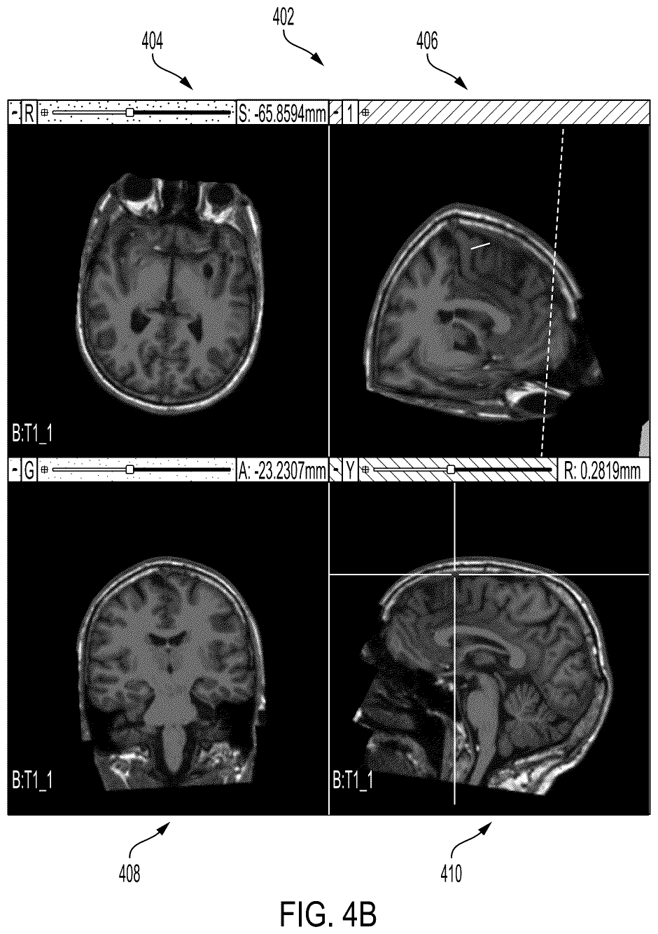

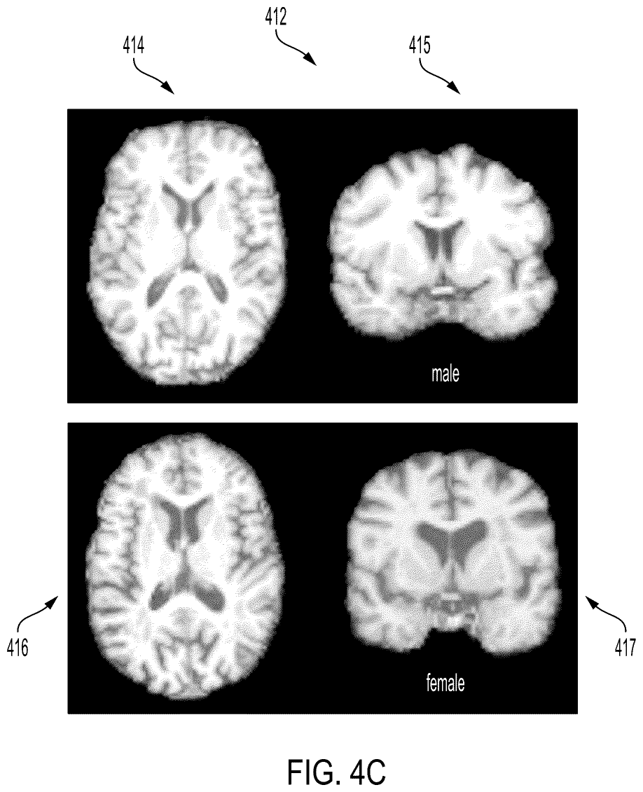

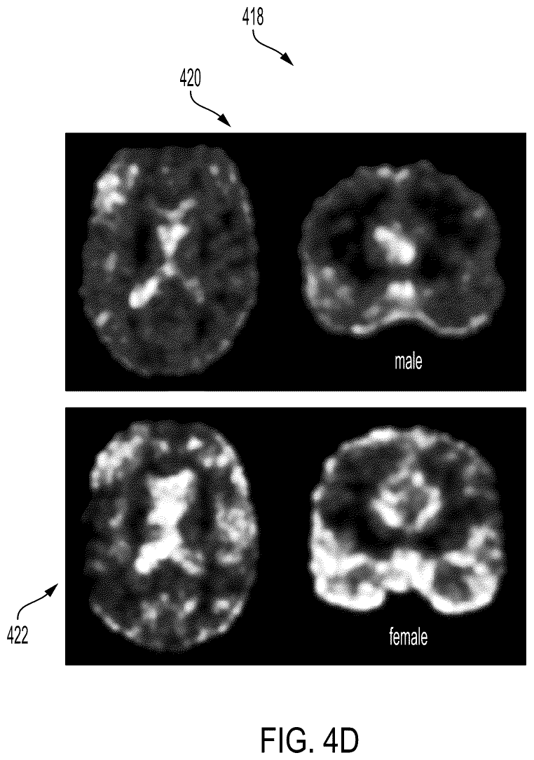

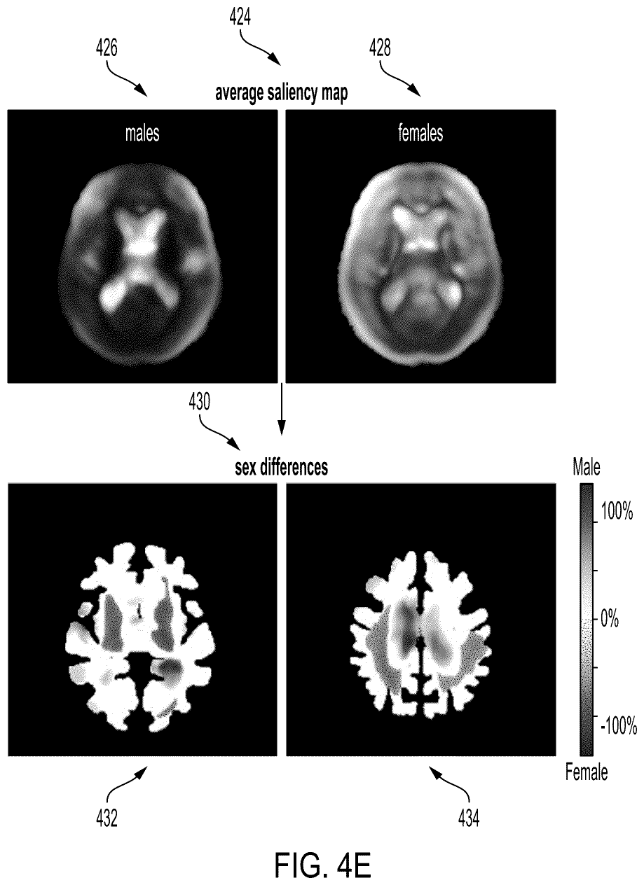

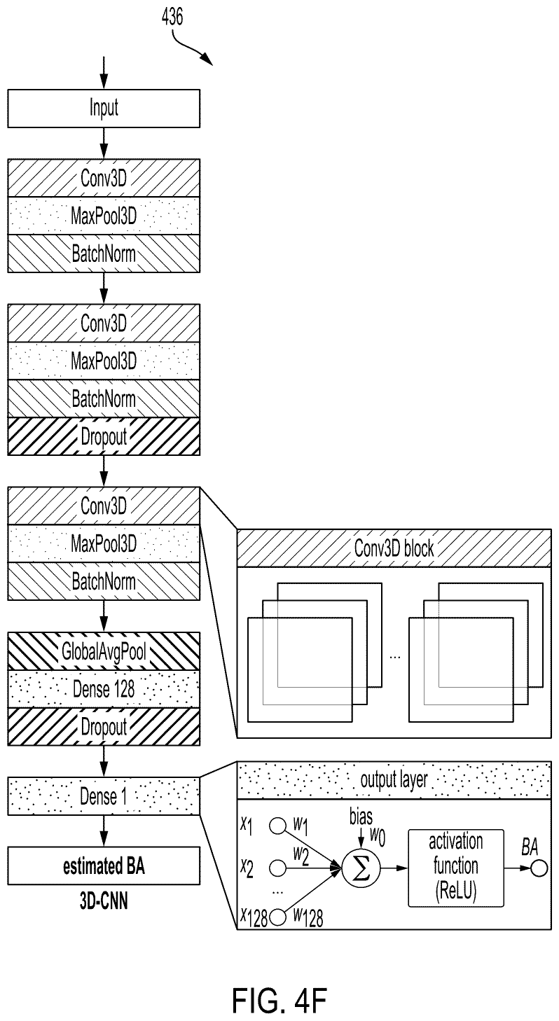

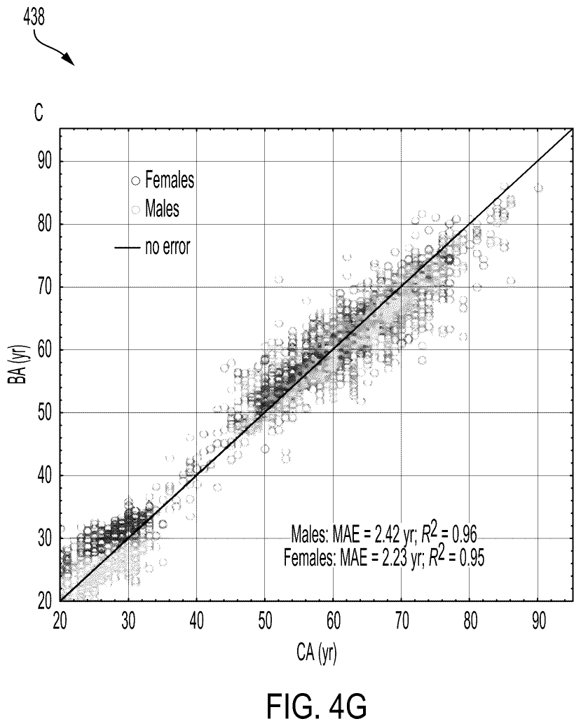

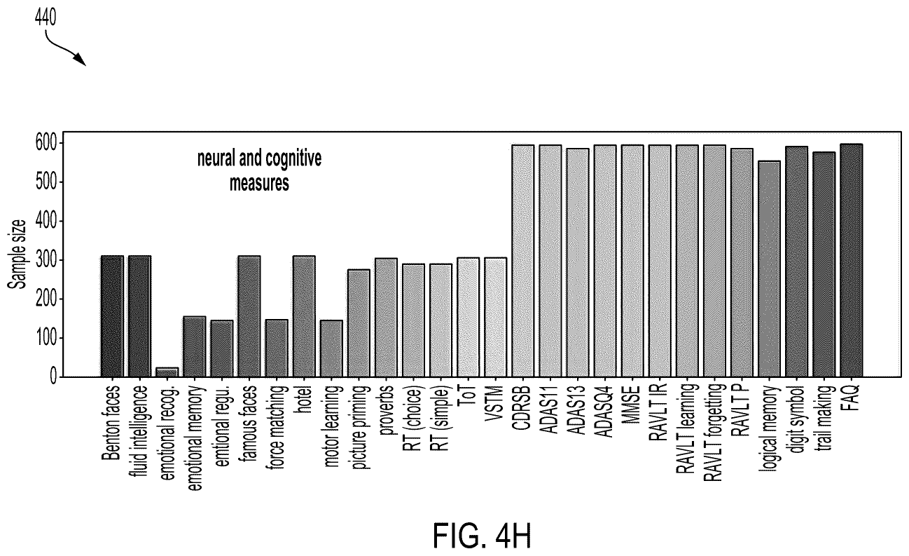

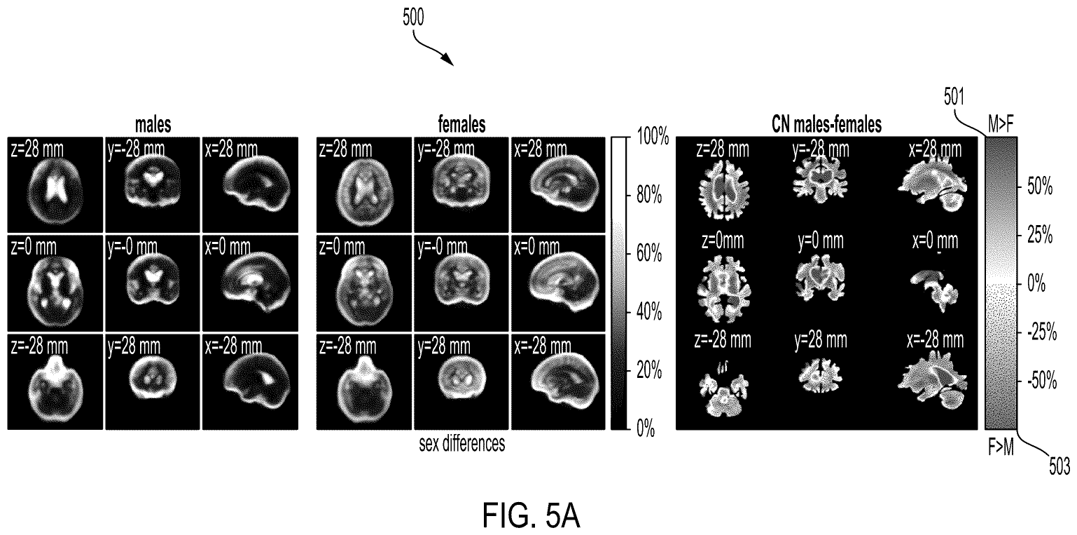

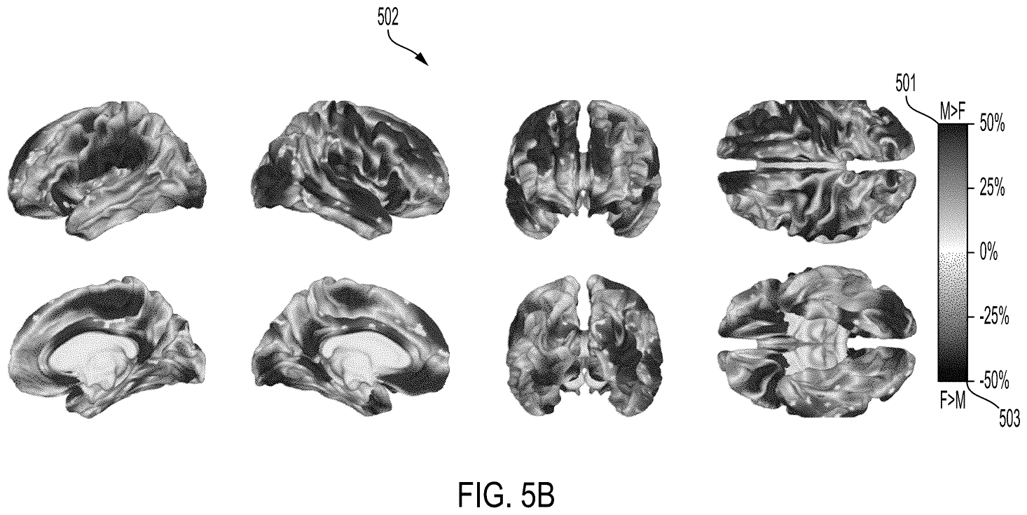

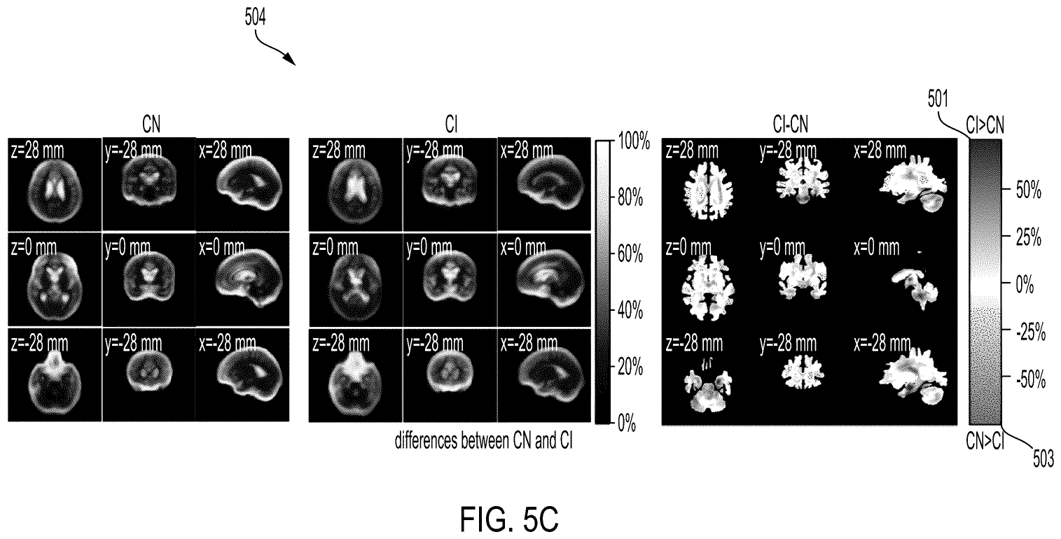

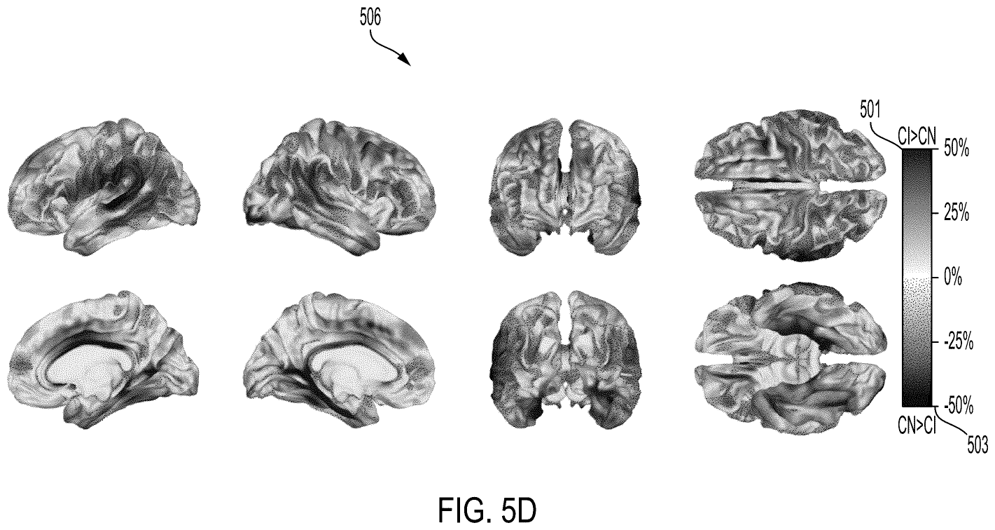

Results are included in the following discussion. With reference to FIGS. 4A-4H, the discussion proceeds first to neuroanatomic patterns of aging. The disclosed system, methods, and apparatus use an interpretable 3D-CNN framework to estimate the BAs of 650 CN adults (age range: 18 to 88 y; 325 males) from the Cambridge Centre for Aging and Neuroscience (CamCAN), as illustrated in FIG. 4A showing aggregate datasets 400 and FIG. 4G showing a chart 438 of BA and CA data. BAs were also estimated in 359 participants with AD dementia (age range: 55 to 92 y; 198 males) and in 351 participants with MCI due to AD (age range 55 to 89; 230 males) from the Alzheimer's Disease Neuroimaging Initiative (ADNI). FIG. 4A shows an example chart of aggregate datasets 400 associated with ADNI. Among participants with MCI, 54% were diagnosed with dementia within 11 y from the acquisition of MRIs analyzed in this study. With reference to FIGS. 4B-4F, aspects are shown that illustrate generation of 3D-CNN saliency maps of each participant's brain to determine how the 3D-CNN weighs each MRI voxel. Saliency maps can help to identify brain locations whose MRI features are weighted more heavily during age estimation (FIGS. 5A-5D). Using this strategy, one may map CI-related aging patterns and study their variation across sexes, brain regions, and subjects, as well as their association with neurocognitive outcome as illustrated in FIG. 4H showing a chart 440.

Turning, then in detail to aspects illustrated in FIGS. 4A-4H, these figures provide an overview of BA estimation by an interpretable 3D-CNN. FIG. 4A provides an aggregate dataset 400 that illustrate the proportions of participants in the aggregate dataset (ADNI, UKBB, CamCAN, and HCP), where each human symbol represents ˜300 participants. FIGS. 4B-E illustrate the following. FIG. 4B shows T₁-weighted MRIs 402, with first MRI 404 and second MRI 406 associated with a male subject and third MRI 408 and fourth MRI 410 associated with a female subject. The MRIs were preprocessed into skull-stripped MRIs shown in FIG. 4C. FIG. 4C illustrates skull-stripped MRIs 412 with first skull-stripped MRI 414 being associated with first MRI 404 (FIG. 4B) and second skull-stripped MRI 415 being associated with second MRI 406 (FIG. 4B) and third skull-stripped MRI 416 being associated with third MRI 408 (FIG. 4B) and fourth skull-stripped MRI 417 being associated with fourth MRI 410. First skull-stripped MRI 414 and second skull-stripped MRI 415 are associated with a male subject and third skull-stripped MRI 416 and fourth skull-stripped MRI 417 are associated with a female subject. Saliency map extraction is performed and, as shown in FIG. 4D, saliency maps 418 are generated with male saliency maps 420 corresponding to first skull stripped MRI 414 (FIG. 4C) and second skull stripped MRI 415 (FIG. 4C) and with female saliency maps 422 corresponding to third skull stripped MRI 416 (FIG. 4C) and fourth skull-stripped MRI 417 (FIG. 4C). Thus, the MRIs are skull-stripped and 3D saliency probability maps were generated from 3D-CNN output for each subject. With reference to FIG. 4E, average saliency maps 424 are generated with a male average saliency map 426 and a female average saliency map 428 being illustrated and with sex differences 430 being illustrated for the male data 432 and for the female data 434. FIG. 4F shows aspects of a processing mechanism 436 that is implemented, and FIG. 4G shows a chart 438 illustrating BA and CA data for males and females. With reference to FIG. 4G, prior to BA estimation using the 3D-CNN, participants were split by sex and assigned randomly into training and test sets. MAE was used to evaluate 3D-CNN performance from BA estimation results for test sets. The test set's CA histogram is displayed. Returning to FIG. 4F of the processing mechanism 436, the 3D-CNN's input consists of T₁-weighted MRIs, and its output are BA estimates. Saliency maps (FIGS. 4D-4F) are extracted from 3D-CNN output after training. A dropout rate of 0.3 is used in all dropout layers, and a ReLU activation function is used in all convolutional and dense layers. xᵢ is the feature map for input i and wᵢ is its weight. FIG. 4H shows a chart 440 of sample sizes for participants with neurocognitive measures.

The results in CN participants (FIGS. 5A-5B) depict illustrations 500, 502 that reveal typical neuroanatomic patterns of aging, including ventricular enlargement, atrophy of frontal, temporal, and hippocampal cortices, and cortical thinning. Cortical features are weighted differently across sexes, which suggests that males' BA estimation is particularly reliant upon Sylvian fissure widening, ventricular enlargement, and cingulate cortex atrophy. Males' BA estimation is also weighted more heavily by features of the lateral temporal lobe and dorsolateral frontal lobe in the right hemisphere, a notable lateralization effect. By contrast, females' saliencies are higher in posterior and medial occipital regions (except the left calcarine sulcus), in the inferior and medial aspects of the parietal lobes, in the supramarginal gyrus and adjacent parietal structures, in the callosal sulcus, in the pars triangularis of the right inferior frontal gyrus, and in posterior insular regions. In females, on average, white matter is weighted more heavily than gray matter when estimating BA.

FIGS. 5C and 5D provide illustrations 504, 506 that facilitate comparison of subject-wise average saliency maps according to the cognitive status (CN vs. CI). This comparison reveals brain features upon which the 3D-CNN relies more when estimating age according to cognitive status. For this reason, such features may reflect how CI modifies regional brain aging. Many structures salient in CN aging are in the cortical gray matter and include the dorsolateral aspect of the right frontal lobe, the lateral aspect of the right temporal lobe, the posterolateral aspect of the right occipital lobe, as well as pericallosal regions in both hemispheres. Cerebral white matter is more salient in aging with CI than in CN aging (FIG. 5C), as is the brainstem, medial aspects of the temporal lobes (including parahippocampal and fusiform gyri), and the caudal portions of the anterior cingulate gyri (FIG. 5D). Appreciable lateralization of saliencies is noted when comparing CN participants to participants with CI, and the lateralization pattern is similar to that obviated by the sex comparison (FIG. 5B). Involved are lateral temporal areas, the angular and supramarginal gyri, middle cingulate cortex, parahippocampal areas, and both medial and dorsolateral prefrontal cortices.

Thus, with reference to FIGS. 5A-5D, a comparison is provided of brain saliency maps across sexes and diagnoses. FIG. 5A provides an illustration 500 of sex-specific mean saliency maps (P_{M}>P_{F}) and the sex dimorphism map ΔP=(P_{M}−P_{F})/[(P_{F}+P_{M})/2] of CN participants. In all cases, canonical cortical views (sagittal, axial, and coronal) are displayed in radiological convention. Higher saliencies (brighter regions) indicate neuroanatomic locations whose voxels contribute more to BA estimation. Regions drawn in the male-associated shade 501 have higher saliencies in males (P_{M}>P_{F}); the reverse (P_{F}>P_{M}) is true for the regions drawn in the female-associated shade 503. FIG. 5B provides an illustration 502 of canonical views of the sex dimorphism map ΔP for CN participants. Sex-specific deviations of AP from its mean across sexes are expressed as percentages of the mean. The male-associated shade 501 indicates that ΔP_{M}>ΔP_{F}, i.e., males have higher saliency; and the female-associated shade 503 indicates the reverse (ΔP_{F}>OP_{M}), i.e., females have higher saliency (FIG. 5C). Like FIG. 5A, for the comparison between CN participants and participants with CI, where ΔP=(P_{C1}−P_{CN})P_{CN}; the male-associated shade 501 indicates P_{C1}>P_{CN}, the female-associated shade 503 indicates P_{CN}>P_{C1}. For FIG. 5D, like FIG. 5B, one may observe the saliency difference AP between CN and CI participants. Images are displayed in radiological orientation convention (the right hand side of the reader is the left hand side of the participant and vice versa).

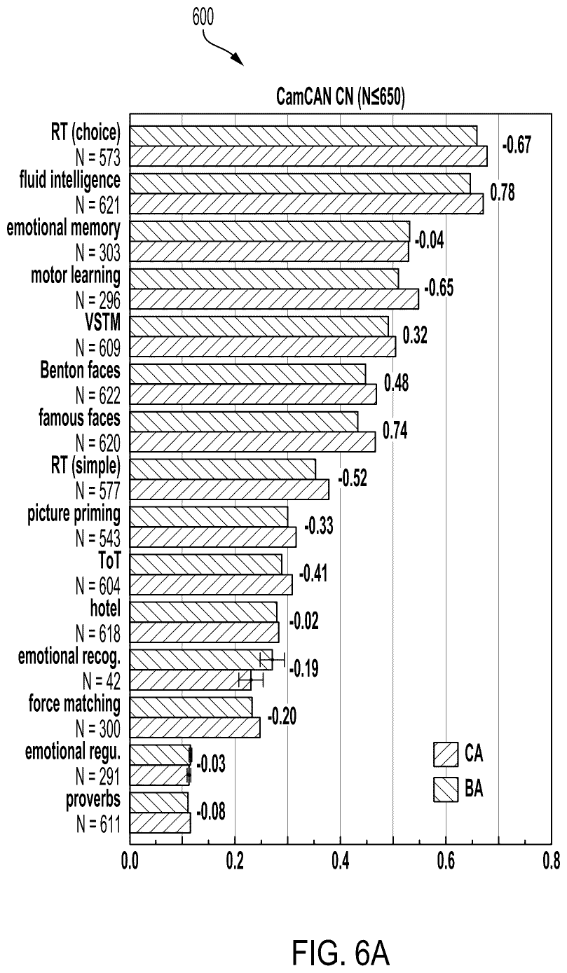

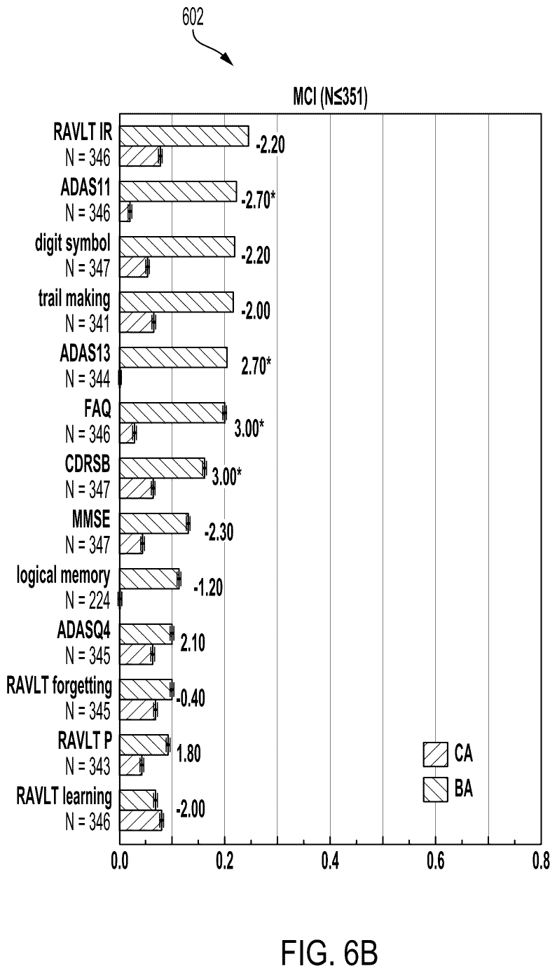

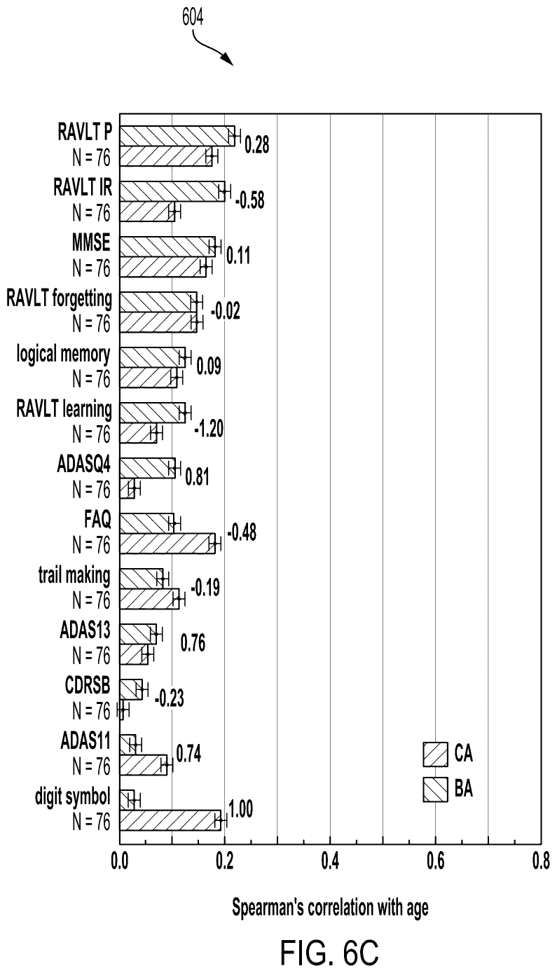

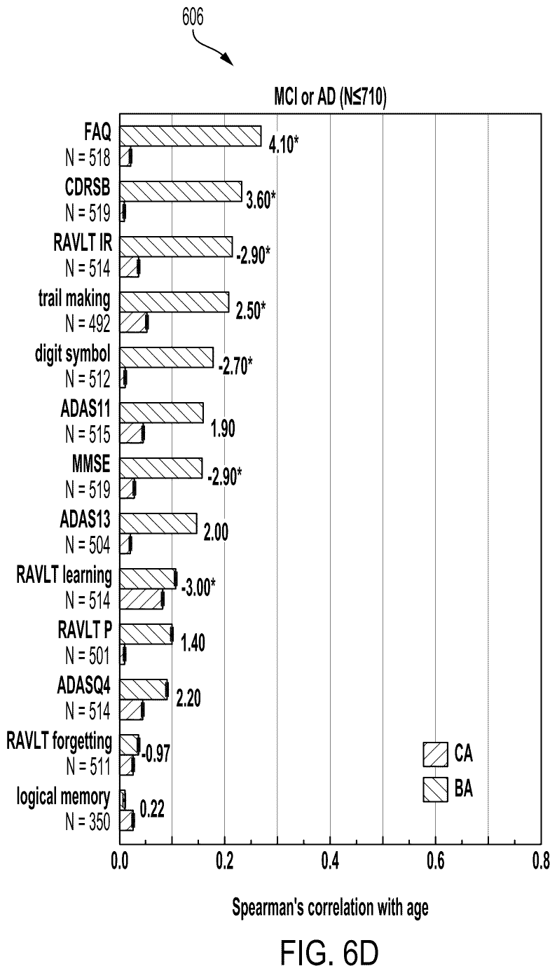

Associations with neurocognitive endophenotypes may now be discussed. The ability of estimated BA to capture neurocognitive endophenotypes was contrasted to that of CA. This was achieved by comparing Spearman's correlations r_{S} between each age (BA and CA) and every neurocognitive measure of CN aging (FIGS. 6A-6D). For all neurocognitive measures, significant r_{S} values reflect typical aging effects on neurocognitive function (worse performance is correlated with older age). As expected, among CN participants, BA and CA reflect cognition to similar extents. For example, among CamCAN CN participants, FIG. 6A illustrates a chart 600 showing that older BA and CA are correlated with worse performance on word finding (picture priming), motor learning (force matching), motor response time (choice and simple response time (RT) tasks), face recognition (Benton's unfamiliar face recognition, famous faces test), Cattell's fluid intelligence, emotional memory, and visual short-term memory (VSTM) measures. In the ADNI CN cohort, FIG. 6B shows a chart 602 that illustrates that no neurocognitive measure examined is significantly more correlated with BA than with CA.

Across participants with CI, BA is significantly more correlated than CA with neurocognitive measures. In participants with MCI, FIG. 6C illustrates a chart 604 showing that older BA (but not CA) is significantly correlated with worse scores on all measures of neurocognitive function examined, except 1) delayed verbal recall and learning on the Rey auditory verbal learning test (RAVLT), 2) delayed word recall measured by the AD assessment scale question 4 (ADAS Q4), and 3) logical memory. For the clinical dementia rating sum of boxes (CDR-SB) and the functional abilities questionnaire (FAQ), the difference in correlations between BA and CA is significant and BA outperforms CA in its ability to reflect neurocognitive function. In participants with AD, no significant correlations exist between BA and any neurocognitive measure apart from FAQ scores. Nevertheless, older CA is correlated with poorer delayed verbal memory (RAVLT forgetting).

By contrast, among all participants with any type of CI (whether MCI or AD), BA (but not CA) is significantly correlated with all measures except delayed verbal recall (RAVLT, ADAS Q4) and logical memory, as illustrated in FIG. 6D showing a chart 606. The difference in correlations between BA and CA is significant for the CDR-SB, mini-mental state exam (MMSE), RAVLT immediate recall (IR), and FAQ. When separating participants with CI by apolipoprotein E4 (APOE4) status, BA is not more correlated with any neurocognitive measure in carriers compared to noncarriers. The omnibus effect of a logistic regression accounting for all interactions between AG, CA, and sex is significant (χ²₃₄₃=29.500, P<0.001). AGs are significantly and positively associated with MCI participants' probability of conversion to AD(β=1.417, t₃₄₃=2.240, P=0.025). The only significant interaction is between AG and sex(β=−1.121, t₃₄₃=−2.129, P=0.033), i.e., MCI females with more negative AGs and MCI males with more positive AGs are significantly more likely to convert to AD. When including all interactions, the omnibus effect of the regression that predicts time to conversion is significant if AG is included as the predictor (R²=0.065, F_{8,181}=2.880, P=0.007) but not if AG is excluded (R²=0.012, F_{4,185}=1.790, P=0.151).

Referring to FIGS. 6A-6D in further detail, including with reference to charts 600, 602, 604, and 606, correlations are shown between neurocognitive measures and both estimated BA and CA. Results are depicted for two independent test sets: CamCAN and ADNI. FIG. 6A displays CN participants from CamCAN, FIG. 6B displays CN participants from ADNI, FIG. 6C displays results only for participants with MCI, and FIG. 6D displays results for participants with either MCI or AD. Referring against to all of FIGS. 6A-6D, for each independent test set, the sample size for each neurocognitive measure is listed below the measure name. Bar charts depict Spearman's correlations r_{S} (along x) between BA (green) or CA (red) and each neurocognitive measure (along y). Bars are contoured in black if r_{S} is significant. Error bar widths equate to one SE of the mean. For each neurocognitive measure, the corresponding bar pair is annotated with Fisher's z statistic. Asterisks indicate neurocognitive measures for which the difference in Spearman's correlations r_{S}(BA)−r_{S}(CA) is significant.

Turning now to a discussion of 3D-CNN benchmarking and evaluation, the discussion will compare the disclosed 3D-CNN to a simple fully convolutional network (SFCN), by replicating its training, validation, and benchmarking. The SFCN was pretrained on 5,698 UK Biobank (UKBB) subjects, whereas the 3D-CNN was trained on 4,681 participants (2,513 females; age range: 22 to 95 y) aggregated across the UKBB, Human Connectome Project-Aging (HCP-A), Human Connectome Project-Young Adult (HCP-YA), and ADNI. In the testing set, the disclosed model's MAE between BA and CA is 2.41 y for males and 2.23 for females. The coefficient of determination R² is 0.96; the correlation coefficient r is 0.98. Across all external testing sets (UKBB, CamCAN, AD, and MCI), the disclosed model has a higher R² than the SFCN.

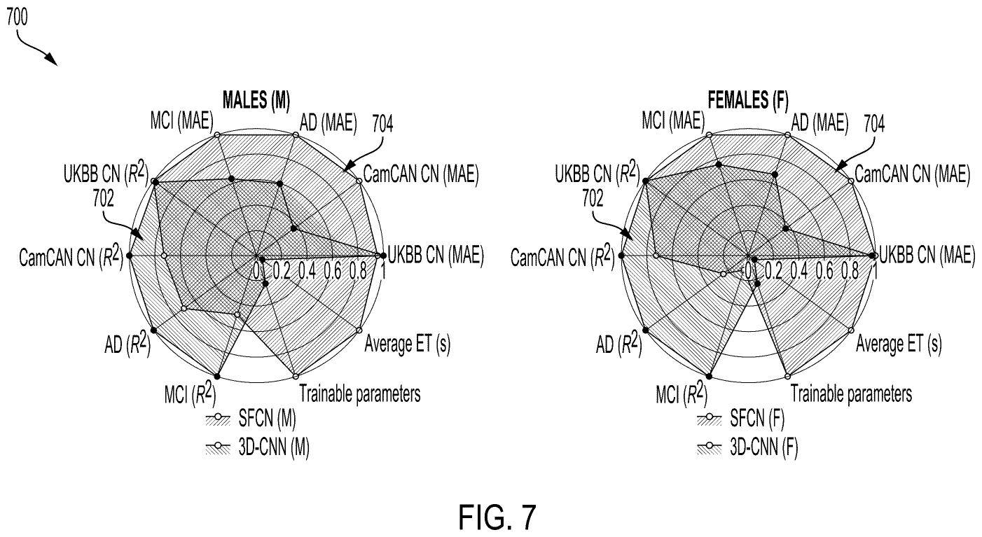

On identical UKBB data (N=518), the 3D-CNN achieves MAEs of 2.27 y (males) and 2.31 y (females), while the SFCN achieves an MAE of 2.14 y across both sexes. In the independent CamCAN CN cohort, the SFCN's MAEs are 9.90 y (males) and 9.17 y (females). By contrast, the disclosed 3D-CNN achieves MAEs of 4.71 y (males) and 3.01 y (females). During pretraining, the SFCN yields an MAE within 2% of the 3D-CNN's. However, in the independent test cohort of CN participants, the MAEs are 42% lower than the SFCN's in the same cohort. The SFCN yields MAEs of 7.72 y (males) and 7.50 y (females) for participants with MCI and 8.24 y (males) and 8.65 y (females) for participants with AD. By contrast, the 3D-CNN model achieves an MAE of 5.26 y (males) and 4.33 y (females) for participants with MCI and 6.48 y (males) and 5.98 y (females) for participants with AD. Compared to the SFCN, the 3D-CNN yields significantly larger mean AGs for A) females with MCI (t₁₄₄=6.595, P<0.001), B) males with AD (t₁₉₅=4.710, P<0.001) and C) females with AD(t₁₆₂=6.200, P<0.001). The 3D-CNN also yields significantly larger AG variances in participants with AD (males: F_{197,197}=1.857, Pitman's t₁₉₆=4.440, P<0.001; females: F_{162,162}=2.493, Pitman's t₁₆₁=6.006, P<0.001). Compared to the 3D-CNN, the SFCN yields significantly larger AG variances for the following CN groups: A) UKBB males (F_{796,796}=1.137, Pitman's t₇₉₅=12.967, P<0.001); B) UKBB females (F_{796,796}=1.097, Pitman's t₇₉₅=9.034, P<0.001); C) CamCAN females (F_{309,309}=7.576, Pitman's t₃₀₈=21.082, P<0.001). As expected, the 3D-CNN's mean AG is ˜75% larger in participants with CI than in CN participants, possibly reflecting faster brain aging in the former. The BA estimation parameters of the 3D-CNN and SFCN, evaluated without fine-tuning, are compared in FIG. 7 showing chart 700. The 3D-CNN has shorter execution times (ETs) and fewer trainable parameters, reflecting lower complexity (FIG. 7). For participants with CI, the disclosed CNN yields higher R² and lower MAEs than the SFCN.

Specifically, FIG. 7 illustrates a chart 700 of radar plots of sex-specific MAEs and performance parameters. Radar plots of MAE, R², and performance parameters (average ET and the number of trainable parameters) according to sex and diagnostic status (CN: UKBB, CamCAN; MCI or AD: ADNI). A SFCN (region 702) is compared to 3D-CNN (region 704). To facilitate simultaneous comparison, all values are normalized to range from 0 to 1, where the maximum value in each measurement was rescaled as 1 and 0 remained as 0.

While biological age can be computed for many phenotypic traits, BA summarizes MRI-derived neuroanatomic profiles using one number. This highlights both the appeal and caveats of this measure. Although straightforward to grasp, BA (as defined here) does not capture the nuances and complexity of brain aging. Nevertheless, with cautious interpretation, BA could assist the diagnosis and prognosis despite its limitations. Early screening for CI can help to monitor and improve the welfare of aging adults. Although positron emission tomography (PET) can aid the diagnosis of AD at the preclinical and prodromal stages, this technique is expensive, involves specialized tracers, and exposes participants to radiation. By contrast, MRI is noninvasive, more affordable, and safer. Thus, MRI-derived BAs that capture neurocognitive decline could become affordable and noninvasive preclinical measures of CI risk.

The correlations of neurocognitive measures with the estimated BAs are, in many cases, significantly stronger than with CA, suggesting that the BAs better reflect neurocognitive functioning. These correlations are critical because one potential utility of BA estimation is to facilitate the early identification of persons at high risk of MCI and AD. AGs are predictive of AD conversion risk. BA is not correlated with neurocognitive function in participants with AD, with the exception of informant-rated functional ability. One possible reason is that the 3D-CNN was trained on CN adults rather than participants with CI. Another reason could be that correlations are more difficult to detect due to lower statistical power (smaller sample size) in participants with AD compared to CI participants. Across persons with CI of any severity, BA (but not (A) is significantly correlated with measures used routinely to screen for (or to diagnose) CI, including MCI (FIGS. 6A-6D). Thus, the contributions herein can help to understand how CI-related neurocognitive changes within specific functional domains reflect neuroanatomic features that modify regional BAs.

The discussion now shifts to sex differences in anatomic brain aging. Of note, for patient-tailored profiling, this approach can generate subject-specific brain saliency maps reflecting individual neuroanatomic patterns of brain aging. Anatomic interpretability of BA is important because 1) brain regions age differently, 2) neuroanatomic alterations with age may reflect distinct disease processes paralleled by BA, and 3) individual neuroanatomic deviations may parallel neurocognitive endophenotypes. Sex differences in saliency confirm findings on the contributions of age to sex dimorphism in the pre- and post-central gyri and the pars triangularis of the left inferior frontal gyrus. Males, who are at higher risk of motor impairment due to Parkinson's disease, exhibit greater saliency in the primary motor cortex. Males' BA estimation relies more on the crowns of gyri on the lateral aspects of the frontal lobes, whereas females' BA estimation relies more on the troughs of sulci. These findings confirm prior reports on sex differences in older adults' cortical gyrification. Males' saliencies are higher along ventricular boundaries, indicating that BAs are disproportionately predicated upon ventricular enlargement in men, as reported elsewhere.

The right hemisphere's higher saliency in males is consistent with their lateralization of language function and with lateralization trends in old age. Thus, in females, typical cortical aging may be relatively slower in the right hemisphere. By contrast, on average, most occipital and medial parietal areas exhibit age-related neuroanatomic patterns that are more salient in males. Males also have higher saliency in superior parietal and frontal regions, reflecting smaller gray matter volumes. By contrast, females have higher saliency at the occipital poles and in occipitoparietal regions, reflecting smaller gray matter volumes in these regions. Females' saliencies are higher across inferior parietal regions, where the cortex is thicker than in males. Thus, the approach to neuroanatomic saliency mapping herein can identify sex differences in the neuroanatomy of cortical aging.

The interpretable 3D-CNN framework captures neuroanatomy changes related to both CN aging and aging with CI. In the case of CN aging, the estimated BAs of CN participants in two independent samples (CamCAN and ADNI) are correlated with neurocognitive measures reflecting typical aging (e.g., motor learning, multitasking, and word finding). In ADNI CN participants, no significant associations were found between neurocognitive measures and either CA or BA. This was expected, as ADNI cognitive measures are sensitive to CI rather than to CN aging. In the case of CI, this work confirms that participants with either MCI or AD have AGs considerably larger than those of sex- and age-matched CN adults, mostly due to older-than-expected brains (BA>CA). Atrophy of the parahippocampal gyrus is a strong structural correlate of MCI and AD; the 3D-CNN's greater reliance on this structure during BA estimation reflects this (FIG. 5C). Similarly, saliency differences between CN and CI participants are greater in parietal, occipital, and temporal cortices (FIG. 5D), whose atrophy is greater in participants with CI and whose burdens of amyloid p plaques and T neurofibrillary tangles are typically higher in AD. The brainstem, which is affected by amyloid deposition early during AD, is more salient in participants with CI than in CN adults. Comparison of the cortical patterns in FIGS. 5B and 5D indicates that saliency differences between sexes are largely paralleled by saliency differences across cognitive statuses (CN vs. CI). This may reflect females' higher risk for AD and supports the hypothesis according to which their higher risk is paralleled by faster cortical aging. Comparison of CN and CI cohorts suggests that the SFCN underestimates mean AG in the latter group and that the expected accuracy of BA estimation is lower for participants with CI. These findings highlight the importance of an accurate BA estimator when studying diseased populations. Some cortical structures that atrophy far more in CI than in CN aging are more salient in the latter (female-associated shade 503 regions in FIG. 5D). This may reflect the fact that the 3D-CNN was trained on a CN adult cohort. During training on this cohort, the 3D-CNN likely relies on features whose variance is moderate in CN aging. When estimating the BAs of participants with CI, however, these features exhibit far greater variability. This may cause their relative saliency to decrease, such that the saliency difference AP between CN and CI aging is negative in such regions. Thus, although features with negative AP values can be useful for understanding how BA estimation relies on CI-related neuroanatomy features, the negative sign of AP must be interpreted cautiously.

In comparing the 3D-CNN to other methods, various beneficial aspects are apparent. The 3D-CNN alleviates major limitations of other approaches. The quantitative comparison below focuses on the SFCN because this open-source approach performed best in a competition for which both training and testing data are available.

Regarding accuracy, the 3D-CNN estimates BA more accurately than the state of the art regardless of whether accuracy is quantified using MAE or R². In the test set, the model yields an MAE of ˜2.3y; this is ˜1 y less than the SFCN, which is the second best. Other (published) BA estimators have MAEs that are even higher than that of the SFCN on their testing data. Presumably, since the MAE is ˜2.3y, these estimators also perform more poorly than the disclosure herein. However, ability to fully ascertain this was limited during testing because there was limited access to the testing sets on which other estimators were benchmarked. These estimators include a best linear unbiased predictor (MAE≃3.3y), a 3D residual neural network (3D-RNN, MAE≃3.3 y), a graph CNN (MAE≃4.6 y), Gaussian process regression (MAE≃4.1 y), support vector regression synergized with a random forest classifier (MAE≃3.5y), and a 3D—DenseNet(MAE≃3.3y).

On the testing set, the model yields R²≃0.96 and r≃0.98. By contrast, the SFCN model yields R²≃0.92 and r≃0.96. During testing, other (published) BA estimators achieve even lower R² than the SFCN. These include Gaussian process regression (R²≃0.91), a 3D-RNN (R²≃0.90) (49), a graph CNN(R²≃0.87) (50), and a 3D-DenseNet (R²≃0.85). The R² of the mechanism disclosed herein is also higher than that of a BA estimator that used an optimized SFCN with R²=0.94. These comparisons suggest that even high R² can involve undesirably large MAE, such that it can be useful to consider both measures when evaluating accuracy.

In all females with CI and in AD males, the AGs herein are significantly larger than those estimated by the SFCN. As expected, the 3D-CNN's estimates of these subjects' CAs are consistently larger than their true CAs. Because CI involves more brain aging, this suggests that the 3D-CNN captures CI better than the SFCN. Females are at higher risk for AD and exhibit faster decline than males. Females already have a larger mean AG in the MCI stage, whereas this is not the case for males until the AD stage. Thus, the model herein captures known sex differences in AD risk.

Variances in AG between the 3D-CNN and SFCN are significantly different for the UKBB cohort even though F=σ_{SFCN}²σ_{CNN}²≃1, which usually implies lack of significant differences in variance. This finding can be explained by use of Pitman's variance ratio test, which is justified here because the variances being compared pertain to correlated samples (CNN and SFCN-computed AGs measured for the same cohort). Because the 3D-CNN and SFCN were both trained on UKBB CN participants, the abilities of these methods to estimate BA for new UKBB participants are likely better (and therefore more similar) than their ability to estimate BA for participants from altogether new cohorts. This similarity may explain the strong correlation r of UKBB BAs across the two methods (females: r=0.989; males: r=0.990). The dependence of Pitman's t on r satisfies t˜(1−r²)^{-1/2}. A Maclaurin series expansion indicates that t→∞ as r→1. Thus, Pitman's t is large when r≃1 even when σ_{SFCN}²/σ_{CNN}²≃1. This explains the power to detect even moderate differences between σ_{SFCN}² and σ_{CNN}² in UKBB CN participants.

Model complexity was quantified using mean ET and the number of trainable parameters in the model. By both measures, 3D-CNN's execution complexity is lower than that of previous approaches. For example, 3D-CNN features a ˜10 times ET compared to the SFCN and ˜4 times fewer trainable parameters. In various instances, other models, such as other models based on the SFCN may have more trainable parameters and may be more challenging to fine-tune. The 3DDenseNet has ˜7 million trainable parameters (compared to the 682,881 of the discussion herein) and requires extensive fine-tuning on new validation datasets via grid searches for optimal hyperparameters.

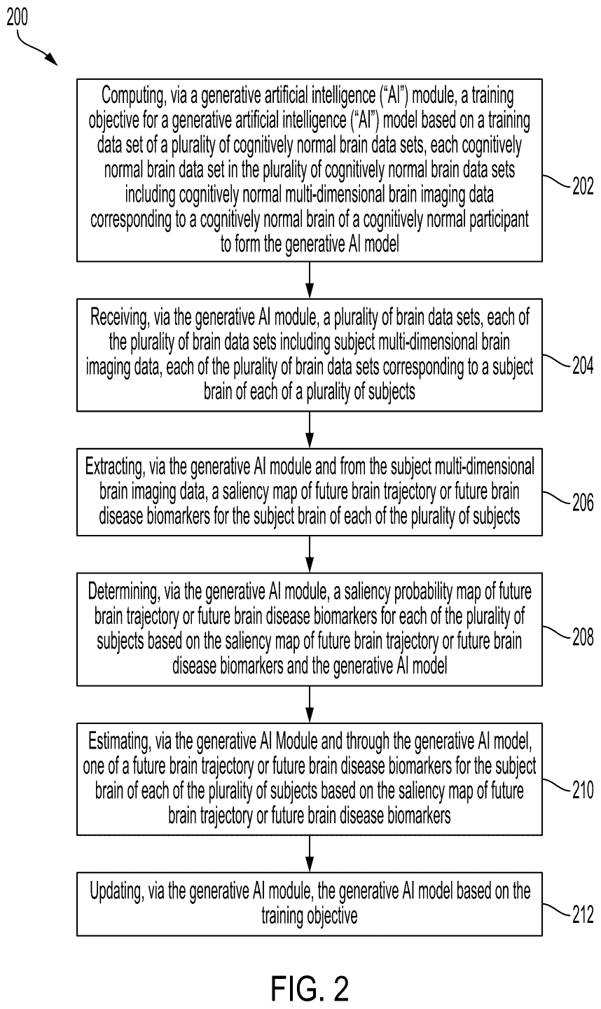

Regarding interpretability, in other instances, a 3D-DenseNet is used to compute saliency by covering the brain with occlusion masks of size 113 mm³=1,331 mm³. The related saliencies may correlate with PET-mapped amyloid R and T burdens. However, for participants with CI, the anatomic patterns of brain aging mapped thereby are broadly similar to those herein (FIGS. 2C and 2D) across similar age ranges. This suggests the hypothesis that BA saliencies such as those herein can reflect dAD-related clinical PET findings. In various instances, one may use two-dimensional (2D) occlusions (box size: 32² mm²=1,024 mm²) to map saliency, whereas in further instances, one may monitor performance and saliency by occluding 3D masks (size: 5³ mm³=125 mm³). The study herein advances the state of the art by 1) providing voxelwise saliency maps to reveal detailed spatial variability at native MRI resolution (1 mm³), 2) reporting comparisons by sex and cognitive status, and 3) conveying how cognitive status relates to neurocognitive function.

Regarding generalizability, most BA estimators are not typically tested across domain-specific neurocognitive measures, whereas the disclosed 3D-CNN features unique generalizability to independent cohorts in its ability to capture neurocognitive endophenotypes. Since the R² values achieved on independent and test data are similar, one may surmise that overfitting was largely avoided. Compared to CA, the BA of participants with any type of CI is significantly more correlated with measures of neurocognitive function routinely used as clinical indicators of CI. Other published approaches have rarely been evaluated according to this critical performance benchmark. Because the 3D-CNN was trained on subjects aged 22 to 95 y, its utility extends across the age range of adulthood.

Although 3D-CNN was validated in cohorts independent from those used for its training, differences in acquisition sequences and scanners across MRIs can affect results. Like other dementia diagnosis criteria, ADNI's have limitations such as a risk of false positive diagnoses, that may affect the findings. Additionally, floor effects may affect cognitive measures in participants with AD by attenuating their correlation. Conceivably, a failure to find significant correlations between BA and neurocognitive measures in participants with AD could be due to lower power to detect small effects in the AD sample, which is smaller (N≤172) compared to the MCI (N≤347) and combined CI (i.e., MCI or AD, N≤519) samples. These nonsignificant correlations, however, are not typically relevant for early CI screening because most participants with severe CI have been already diagnosed by the time brain MRIs are typically acquired. The nonuniform distribution of CAs in aggregate sample translates into potential training data imbalance and inaccuracy in BA estimates. Nevertheless, the approach is more accurate than others currently available, as reflected by the test set's MAE and R², which are the lowest reported to date. Due to the lack of ground truth, there is no consensus on how the interpretability of various approaches ought to be evaluated.

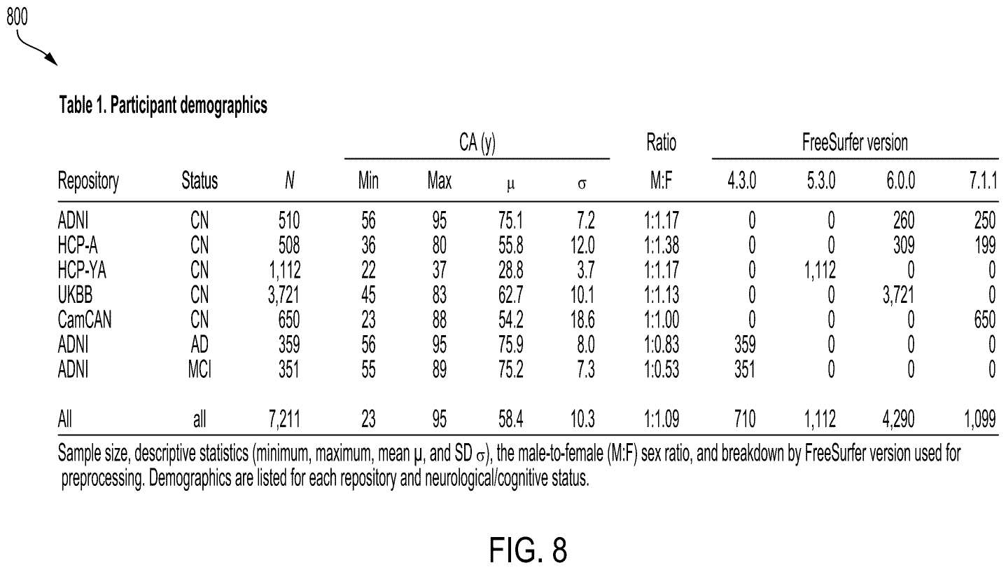

Turning now to methods, and specifically, participants and neuroimaging, the aggregate dataset consists of 5,851 CN individuals (3,142 females) aged 22 to 95 yr sampled from ADNI (N=510), HCP-A (N=508), HCP YA (N=1,112), and UKBB(N=3,721) (see FIG. 8 illustrating table 800). The primary goal of ADNI has been to test whether serial MRI, positron emission tomography, other biological markers, and clinical and neuropsychological assessment can be combined to measure the progression of MCI and early AD. For UKBB data, the discussion may use preprocessed images generated by a UKBB pipeline whose output included FreeSurfer reconstructions.

An independent test set of 650 CN participants aged 18 to 88 yr was obtained from CamCAN. Additionally, 408 participants with MCI and 359 participants with AD were obtained from ADNI (see FIG. 8 illustrating table 800). CamCAN inclusion/exclusion criteria and ADNI eligibility/diagnosis criteria are described elsewhere. N=75 participants with MCl were excluded due to MCl diagnosis being unrelated to AD, leaving 351 participants with MCI (190 converted to AD, 161 did not). Of 524 CI (MCI or AD) participants whose correlations between BA and neurocognitive scores were computed, 307 participants were APOE4 carriers.

Neurocognitive measures available in CamCAN and ADNI were used to evaluate the utility of estimated BAs to capture neurocognitive phenotypes. Thirteen cognitive measures that assess emotional processing, executive function, memory, and motor function were obtained from the CamCAN repository. Emotional processing was measured via 1) Ekman's emotion expression recognition test, 2) the emotional memory test, and 3) the emotional regulation test. Executive function was measured using 1) Cattell's fluid intelligence test, 2) the hotel test, and 3) a proverb comprehension task. Memory was measured using 1) Benton's face recognition test, 2) the famous faces test, 3) a picture priming task, 4) the tip of the tongue (ToT) test, and 5) a VSTM task. Motor function was assessed via 1) a force matching task, 2) a motor learning task, 3) a reaction time (RT) “choice” task, and 4) a RT “simple” task.

Nine cognitive measures that assess neural function, cognitive performance, and functional impairment were obtained from the ADNI repository. To eliminate systematic variability in FreeSurfer software versions, this discussion limited correlation analysis for the Cl cohort to subjects from ADNl₁ only (FreeSurfer v4.3). For neural function, four established dementia rating scales were obtained, including 1) the clinical dementia rating scale-sum of boxes (CDR-SB), 2) the diagnostic ADAS versions 11 and 13, and 3) the MMSE. Cognitive performance was measured via four neuropsychological measures: 1) the RAVLT, 2) delayed recall on the logical memory test, 3) the digit symbol substitution test, and 4) the trail-making test. Functional impairment was measured by the FAQ.

Regarding MRI preprocessing, Freesurfer's recon-all function was used to reconstruct and segment T₁-weighted MRIs. This process includes skull-stripping, motion correction, normalization of nonuniform signal intensities, Talairach space transformation, removal of nonbrain tissues, and registration of all subjects' brains into a common coordinate space. FreeSurfer (FS) was used for at least three reasons: 1) UKBB makes FS reconstructions available; 2) the FS workflow is fully automated and thus convenient; 3) the study involves surface analyses and registrations across native and atlas spaces, which FS facilitates. During FS preprocessing using recon-all, all MRIs were affinely registered to the MNI₃₀₅ atlas. Due to sourcing from several MRI repositories, enhancement of segmentation accuracy differed slightly between cohorts. UKBB and HCP-YA reconstructions were enhanced using T₂-weighted MRIs, while ADNI, HCP-A, and CamCAN were enhanced using fluid-attenuated inversion recovery MRIs.

A DL regression model was constructed using a 3D-CNN whose inputs are FS brain.mgz output files and whose outputs are estimated BAs. The DL architecture was implemented in Python 3.6 using TensorFlow 2.7.0 and executed on a computer with an Intel Core i7 processor (2.2 GHz clock speed) with 16 GB of RAM and a 12 GB NVIDIA Tesla K80 graphical processing unit. The 3D-CNN includes three convolutional blocks followed by two dense layers. The input matrix size is 82×86×100. Each convolutional (conv) block has a 3D conv layer, a batch normalization layer, a max-pooling layer, and an optional dropout layer. The filter sizes of the first three (conv) blocks are 64, 128, and 128, respectively. Conv block filter size determines the dimensionality of the output space. The rectified linear unit (ReLU) activation function is applied to all conv and dense layers. The ReLU activation function is defined as g(x)=max(0, x) for input x. g(x) can efficiently reduce the likelihood of a vanishing gradient and makes the output more sparse. After the conv blocks, the fourth block consists of one global average pooling layer (used for global average pooling of 3D data), one dense layer, and one dropout layer (dropout rate=0.3). The resulting feature map, of size 18×18×18×128, is pooled to 128×1 and then projected onto the output dense layer, which has one output neuron to estimate BA using regression.

One may choose MSE as loss function and use an Adam optimizer (learning rate=0.001). The advantage of outputting BAs as real numbers rather than assigning them to discrete age bins is that, in the former case, BA outputs are assigned within a continuous domain and range. Due to regression to the mean, estimated BAs exhibit a previously documented CA-dependent bias. To alleviate this effect, one may use a zero correlation constraint method to regress out the bias from the BAs of testing set participants. This is done separately for each cohort. Bias-corrected BAs are used for all analyses. CN participants are aggregated from UKBB, HCP-A, HCP-YA, and ADNI. Participants may be randomly assigned into training and test sets of sizes equal to 20% of the total sample size (N=5,851).

For, 3D-CNN training and testing the CNN architecture was optimized and hyperparameters finetuned. 2D-CNNs use 2D kernels to estimate sliding windows across single slices, such that leveraging information from adjacent slices is challenging. This disclosure therefore chose a 3D-CNN that overcomes this deficit by using 3D kernels to estimate sliding windows for volumetric patches. The latter captures interslice image context and improves the model performance. This disclosure also included dropout and batch normalization layers because these help to alleviate overfitting. Grid and random searches determined suitable hyperparameter values (e.g., batch size, kernel size, weight decay). An n dimensional grid was defined to map the n hyperparameters and to identify their ranges. This disclosure examined all possible 3D-CNN configurations to identify optimal values for each hyperparameter. Since this discussion uses MSE as a loss function, one may select a configuration with the lowest loss value (error).

The 3D-CNN was tested on independent cohorts to refine the 3D-CNN architecture, illustrate model generalizability, alleviate data overfitting, and compare the 3D-CNN to other approaches. The testing set was designed to include a random selection of participants from the same cohorts as the training set. To avoid overfitting the 3D-CNN to the training set, the performance was monitored on the testing set. To avoid overfitting on both training and testing sets, one may test the model on two independent cohorts (CamCAN and ADNI) that have not been used for the 3D-CNN design. The latter of these cohorts included participants with a range of cognitive statuses (CN, MCI, or AD).

After computing AGs for identical samples using both 3D-CNN and the SFCN, one may perform Welch's t-tests for paired samples with unequal variances to compare the mean AGs obtained using the two methods. AG variances were compared using Pitman's variance ratio test for correlated samples, whereby

$$F = \frac{{σ}_{1}^{2}}{{σ}_{2}^{2}} , Pitman ’ s {t}_{N - 2} = \frac{[ ( F - 1 ) \sqrt{N - 2} ]}{[ 2 \sqrt{F ( 1 - {r}^{2} )} ]} ,$$

and r is the correlation of AG_{SFCN} with AG_{CNN}. The AG variances are σ₁ and σ₂, whose subscripts {1,2} denote the SFCN or CNN, as needed, to satisfy the inequality σ₁>σ₂.

The disclosure continues with a discussion of BA associations with sex and neurocognition. Each neurocognitive measure m was obtained from CN participants (ADNI and CamCAN) and from participants with MCI or AD (ADNI). These measures were not normally distributed, so their Spearman rank correlations r_{S}((m, BA) and r_{S}((m, CA) were computed. These correlations were compared using Fisher's two-sided z-test after multiple comparison correction using the Benjamini-Hochberg procedure (false discovery rate=0.05). |r_{S}| and |z| were also calculated for measures whose lower scores indicate better performance. Test statistics and their degrees of freedom, confidence intervals, and effect sizes were tabulated (SI Appendix, Tables S1S5). A logistic regression examined whether AG, CA, sex, and their interactions predicted the probability of conversion from MCI to AD. Another linear regression (independent variables: CA, sex, and their interactions) evaluated the ability of AG to predict the interval between MRI acquisition and AD conversion. AG and its interactions were added to this (reduced) model to examine how AG altered the significance of the regression.

A saliency map is a topographically organized depiction of the visual saliency in an MRI volume V₀. Here, the discussion extends a saliency approach for 2D-CNNs to the 3D case. For an MRI brain volume V₀ and a 3D-CNN model with score function S(V), rank voxels in V₀ based on their importance to S(V). Consider the linear score model S(V)=w^{T}V+b, where the volume V, weight w, and bias b are in one-dimensional (vectorized) forms. Since the 3D-CNN and score function are highly nonlinear functions of V, the linear score model cannot be applied directly. Approximate S(V) at V₀ using the first-order Taylor series S(V₀)≃w₀^{T}V₀+b₀, where w₀=∂S/∂V|V₀ is the partial derivative of S(V) at V₀ and b₀=b|V₀ is the bias b at V₀. The spatial and temporal distributions of saliencies contain unique patterns conveying information about BA.

Regarding, saliency associations with sex and neurocognition, two distinct workflows were used for volume- and surface-level transforms, respectively, to remove the confounding effects of subject differences in brain shape and size. For volume level analysis, each saliency map was nonlinearly registered to the FS fsaverage atlas. To this end, T₁-weighted brain volumes were first registered to the atlas in MATLAB using the imregister function, which applied the transformation from native space to the atlas, as provided by FS. MATLAB's imregdemons function was used to deform nonlinearly and to map T₁-weighted scans onto the atlas. The transformations above were applied to each subject's saliency map, resulting in registration to the atlas. For surface-level analysis, saliencies were projected to the native cortical surface. To achieve this, each subject's saliency was projected onto the cortical mantle as a cortical overlay using a customized algorithm for volume-to-surface mapping. Briefly, voxels assigned to the gray matter ribbon by FS were considered. At each vertex of the native mesh for the mid-thickness surface, ribbon voxels were selected within a cylinder that lay orthogonally with respect to the local surface. The cylinder was centered on the vertex and height and radius were equal to the local cortical thickness. The saliency of ribbon voxels within the cylinder was averaged according to a Gaussian weighted function (full width at half maximum=4 mm, σ=5/3 mm) to compute a mean saliency value at the surface vertex in question. After cortical surface projection, each subject's saliency overlay was registered from native space onto the atlas. Subjects' saliency probability overlays were averaged into a cortical map of mean saliency.

For both volume- and surface-level analyses, each saliency map M was operationalized into a saliency probability map P by dividing saliency at each brain location by the sum of all brain saliencies. An average saliency probability map was computed for each sex and cognitive status, yielding P_{M} for males, P_{F} for females, P_{CN} for CN adults, and P_{C1} for participants with any form of CI. Both P_{CN} and P_{C1} were computed after averaging across sex effects. Relative sex differences in P were computed as (P_{M}−P_{F})/[(P_{F}+P_{M})/2], i.e., as sex-specific deviations from the average across sexes. The relative deviation of participants with CI from CN participants was computed as (P_{C1}−P_{CN})/P_{CN}. Relative saliency differences between sexes or diagnostic statuses were mapped after thresholding to include only statistically significant values. For each salience value considered, significance was evaluated using a paired-sample t-test (α=0.05). Results were corrected for multiple comparisons using the Benjamini-Hochberg procedure (false discovery rate=0.05).

For volume-level visualization, CN participants' mean saliency maps were plotted for each sex along the coronal (x), sagittal (y), and axial (z) planes. For each coordinate, maps were generated along planes whose equations were specified by coordinate values of −28 mm, 0 mm, and 28 mm, respectively. In CN participants and participants with CI, the procedure was repeated after averaging across sexes. For surface-level visualization, gray matter saliencies were mapped onto the cortex to compare different cortical locations relative importance to the 3D-CNN when estimating BA.

3D-CNN software is available from https://github.com/irimia-laboratory/USC_BA_estimator MRI data are publicly available from ADNI (https://adni.Ioni.usc.edu/), UKBB (https://www.ukbiobank.ac.uk/), CamCAN (https://www.cam-can.org/), and HCP (https://www.humanconnectome.org/). Data used in the preparation of this work was obtained from the Alzheimer's Disease Neuroimaging Initiative (ADNI) database (adni.loni.usc.edu).

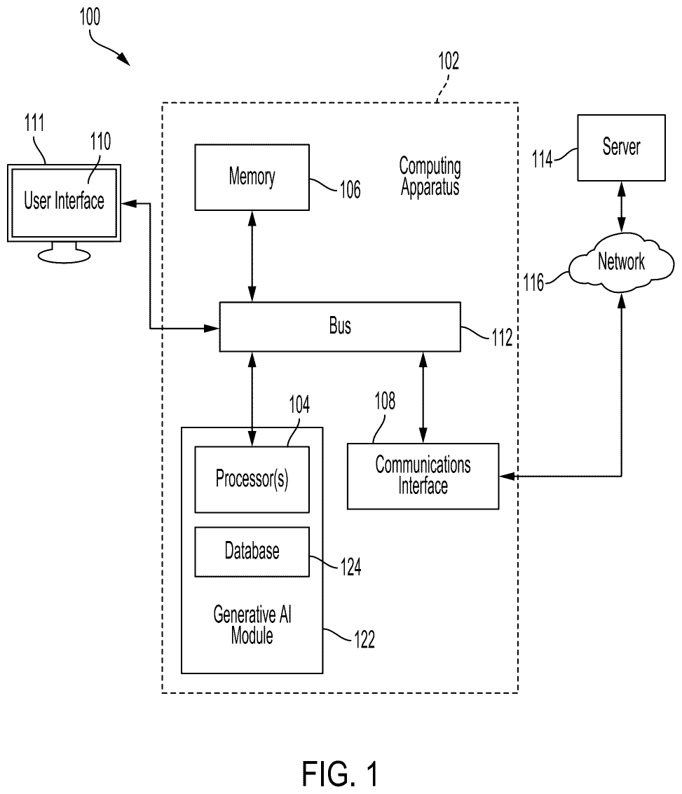

Having introduced various aspects of systems, methods, and devices, now is a convenient time to discuss a few example embodiments thereof. Referring now to FIG. 1, a system 100 (e.g., a computing system) for personalized profiling of future brain trajectories using generative artificial intelligence is disclosed herein. The system 100 (e.g., a computing system) may include a computing apparatus 102. The computing apparatus 102 may include one or more processors 104, a memory 106 and/or a bus 112 and/or other mechanisms for communicating between the one or more processors 104. The system 100 may be a cloud computing system including processors, servers, storage, databases, networking, software, analytics, and/or intelligence accessed or performed over or using the Internet (“the cloud”). The one or more processors 104 may be implemented as a single processor or as multiple processors. The one or more processors 104 may execute instructions stored in the memory 106 to implement the applications of the system 100.

The one or more processors 104 may be coupled to the memory 106. The memory 106 may include one or more of a Random Access Memory (RAM) or other volatile or non-volatile memory. The memory 106 may be a non-transitory memory or a data storage device, such as a hard disk drive, a solid-state disk drive, a hybrid disk drive, or other appropriate data storage, and may further store machine-readable instructions, which may be loaded and executed by the one or more processors 104.

The memory 106 may include one or more of random-access memory (“RAM”), static memory, cache, flash memory and any other suitable type of storage device or computer readable storage medium, which is used for storing instructions to be executed by the one or more processors 104. The storage device or the computer readable storage medium may be a read only memory (“ROM”), flash memory, and/or memory card, which may be coupled to a bus 112 or other communication mechanism. The storage device may be a mass storage device, such as a magnetic disk, optical disk, and/or flash disk that may be directly or indirectly, temporarily, or semi-permanently coupled to the bus 112 or other communication mechanism. The storage device may be electrically coupled to some or all of the other components within the system 100 including the memory 106, the user interface 110 and/or the communication interface 108 via the bus 112. In various embodiments, the user interface 110 is configured to be displayed on a display device 111 (e.g., a monitor, a tablet, a phone, etc.).

The term “computer-readable medium” is used to define any medium that can store and provide instructions and other data to a processor, particularly where the instructions are to be executed by a processor and/or other peripheral of the processing system. Such medium can include non-volatile storage, volatile storage, and transmission media. Non-volatile storage may be embodied on media such as optical or magnetic disks. Storage may be provided locally and in physical proximity to a processor or remotely, typically by use of network connection. Non-volatile storage may be removable from computing system, as in storage or memory cards or sticks that can be easily connected or disconnected from a computer using a standard interface.

The system 100 may include a user interface 110. The user interface 110 may include an input/output device (e.g., a display device 111). The input/output device may receive user input, such as a user interface element, hand-held controller that provides tactile/proprioceptive feedback, a button, a dial, a microphone, a keyboard, or a touch screen, and/or provides output, such as a display, a speaker, an audio and/or visual indicator, or a refreshable braille display. The display device 111 may be a computer display, a tablet display, a mobile phone display, an augmented reality display or a virtual reality headset. The display device 111 may output or provide a data related to personalized spatial profiling of cognitive or affective saliency on brain structure and aging using machine intelligence.

The user interface 110 may include an input/output device that receives user input, such as a user interface element, a button, a dial, a microphone, a keyboard, or a touch screen, and/or provides output, such as a display, a speaker, headphones, an audio and/or visual indicator, a device that provides tactile/proprioceptive feedback or a refreshable braille display. The speaker may be used to output audio associated with the audio conference and/or the video conference. The user interface 110 may receive user input that may include configuration settings for one or more user preferences, such as a selection of joining an audio conference or a video conference when both options are available, for example.

The system 100 may have a network 116 connected to a server 114. The network 116 may be a local area network (LAN), a wide area network (WAN), a cellular network, the Internet, or combination thereof, that connects, couples and/or otherwise communicates between the various components of the system 100 with the server 114. The server 114 may be a remote computing device or system that includes a memory, a processor and/or a network access device coupled together via a bus. The server 114 may be a computer in a network that is used to provide services, such as accessing files or sharing peripherals, to other computers in the network.

The system 100 may include a communication interface 108, such as a network access device. The communication interface 108 may include a communication port or channel, such as one or more of a Dedicated Short-Range Communication (DSRC) unit, a Wi-Fi unit, a Bluetooth® unit, a radio frequency identification (RFID) tag or reader, or a cellular network unit for accessing a cellular network (such as 3G, 4G or 5G). The communication interface may transmit data to and receive data from the different components.

The server 114 may include a database. A database is any collection of pieces of information that is organized for search and retrieval, such as by a computer, and the database may be organized in tables, schemas, queries, reports, or any other data structures. A database may use any number of database management systems. The information may include real-time information, periodically updated information, or user-inputted information.

In various embodiments, the computing apparatus 102 can include a generative artificial intelligence (“AI”) module 122. The generative AI module 122 can include the one or more processors 104. Stated another way, the generative AI module 122 can be run, or operated by the one or more processors 104. In various embodiments, the generative AI module 122 can perform the steps of the methods claimed herein and output an estimated cognitive or affective saliency to the user interface 110.

In various embodiments, the generative AI module 122 is configured to compute a training objective based on training data from a database 124 as described further herein. In this regard, the generative AI module 122 is configured to update a generative AI model based on the training objective and data received from the database 124. In various embodiments, the training objective of the generative AI module 122, as described further herein can include a future brain trajectory, one or more future brain disease biomarkers, or the like.

In various embodiments, the generative AI module 122 includes Generative Adversarial Networks (GAN) and/or Variational Autoencoders (VAEs). In this regard, in a GAN model, two machine learning modules can be trained simultaneously, a generator and a discriminator. The generator can create new outputs that resemble the training data, whereas the discriminator can evaluate the generated data and provide feedback to the generator to improve its outputs. In various embodiments, in a VAE model, the generative AI module 122 can utilize a single machine learning model that is trained to encode data into a low-dimensional representation that captures the data's important features, structure, and relationships in a smaller number of dimensions. The model then decodes the low-dimensional representation back into the original data. Essentially, the encoding and decoding processes allow the model to learn a compact representation of the data distribution, which it can then use to generate new output. In various embodiments, the generative AI module 122 is configured to generate new outputs that resemble the training data. In various embodiments, the training data can include templates.

Referring now to FIG. 2, a flow chart of a process 200 capable of being performed by the system 100 of FIG. 1 is illustrated, in accordance with various embodiments. In various embodiments, the process 200 comprises computing, via a generative artificial intelligence (“AI”) module 122, a training objective for a generative artificial intelligence (“AI”) model based on a training data set of a plurality of cognitively normal brain data sets, each cognitively normal brain data set in the plurality of cognitively normal brain data sets including cognitively normal multi-dimensional brain imaging data corresponding to a cognitively normal brain of a cognitively normal participant to form the generative AI model (step 202). The process 200 comprises receiving, via the generative AI module 122, a plurality of brain data sets, each of the plurality of brain data sets including subject multi-dimensional brain imaging data, each of the plurality of brain data sets corresponding to a subject brain of each of a plurality of subjects (step 204). The process 200 comprises extracting, via the generative AI module 122 and from the subject multi-dimensional brain imaging data, a saliency map of future brain trajectory or future brain disease biomarkers for the subject brain of each of the plurality of subjects (step 206). The process 200 comprises determining, via the generative AI module 122, a saliency probability map of future brain trajectory or future brain disease biomarkers for each of the plurality of subjects based on the saliency map of future brain trajectory or future brain disease biomarkers and the generative AI model (step 208). The process 200 comprises estimating, via the generative AI module 122 and through the generative AI model, one of a future brain trajectory or future brain disease biomarkers for the subject brain of each of the plurality of subjects based on the saliency map of future brain trajectory or future brain disease biomarkers (step 210). The process 200 comprises updating, via the generative AI module 122, the generative AI model based on the training objective (step 212).

In various embodiments, the training objective of generative AI model includes a generative neural network or ensemble of generative, autoencoder, or diffusion neural networks.

In various embodiments, the subject multi-dimensional brain imaging data is a T1-weighted, T2-weighted, diffusion-weighted, fMRI-weighted, or FLAIR-weighted magnetic resonance image.

In various embodiments, determining the saliency probability map of future brain trajectory, including brain aging trajectory saliency and disease biomarker trajectory saliency, for the subject brain of each of the plurality of subjects includes calculating an average of the saliency probability map of future brain trajectory or future brain disease biomarkers based on a deviation from the training data set. In various embodiments, determining the average trajectory map, including brain aging trajectory map and disease biomarker trajectory map, for the subject brain of each subject in the plurality of subjects includes calculating the average future brain trajectory map, including brain aging trajectory map and disease biomarker trajectory map.

In various embodiments, the saliency probability map of future brain trajectory, including brain aging trajectory saliency and disease biomarker trajectory saliency, is determined by dividing the saliency probability map of future brain trajectory or future brain disease biomarkers at each of a plurality of brain locations by a sum of all brain saliencies.

In various embodiments, the cognitively normal multi-dimensional brain imaging data is generated by pre-processing magnetic resonance image(s) for skull-stripping and registering of the cognitively normal brain of the cognitively normal participant into a common coordinate space.

In various embodiments, the process 200 further comprises analyzing, at a surface level, the saliency probability map of future brain trajectory, including brain aging trajectory and disease biomarker trajectory, for the subject brain of each of the plurality of subjects. The process 200 may further include analyzing, at a volume level, the saliency probability map of future brain trajectory, including the brain aging trajectory and disease biomarker trajectory, for the subject brain of each of the plurality of subjects. In various embodiments, in response to the analyzing at the surface level and the analyzing at the volume level, confounding effects of differences in brain shape and size are removed. In various embodiments, at the surface level, saliencies from the saliency probability map of future brain trajectory (e.g., a cognitive or affective saliency map), including the brain aging trajectory and disease biomarker trajectory, are projected to a native cortical surface. In various embodiments, at the surface level, saliencies from the saliency probability map of future brain trajectory, including the brain aging trajectory and disease biomarker trajectory, are projected onto a cortical mantle as a cortical overlay by volume to surface mapping. In various embodiments, at the surface level a mean value for the saliency map of future brain trajectory, including the brain aging trajectory and disease biomarker trajectory, is calculated at a surface vertex by averaging the saliency map of future brain trajectory across cortical ribbon voxels within a cylinder according to a Gaussian weighted function. In various embodiments, the saliency probability map of future brain trajectory, including the brain aging trajectory and disease biomarker trajectory, determined for the subject brain of each of the plurality of subjects is calculated by dividing the saliency probability map of future brain trajectory or future brain disease biomarkers at each of a plurality of brain locations by a sum of all brain saliencies at both the surface level and the volume level.

In various embodiments, the process 200 further comprises transmitting, via the generative AI module 122, the saliency probability map of future brain trajectory, including brain aging trajectory and disease biomarker trajectory, for the subject brain of each of the plurality of subjects to a display device 111.

In various embodiments, the saliency probability map of future brain trajectory, including the brain aging trajectory and disease biomarker trajectory, for the subject brain of each of the plurality of subjects is displayed on the display device 111 in response to the transmitting.

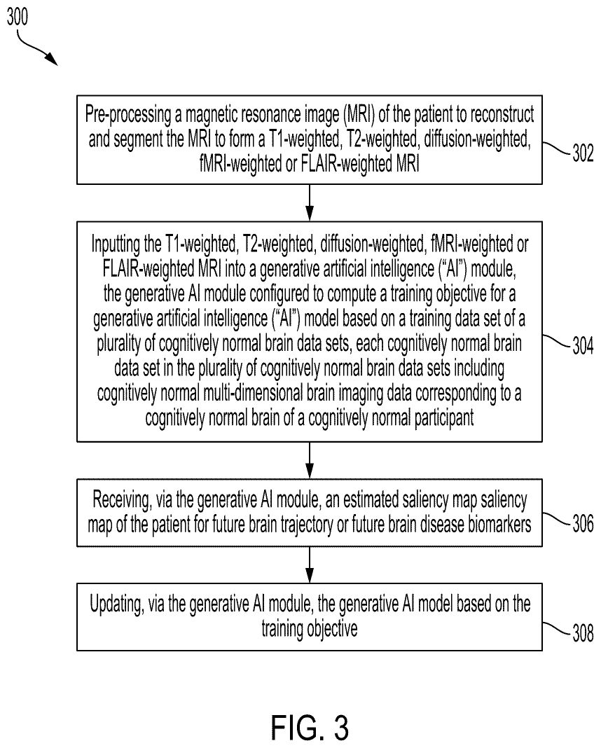

Referring now to FIG. 3, a method 300 of estimating a saliency probability map of future brain trajectory, including brain aging trajectory and disease biomarker trajectory, of a patient, is illustrated. The method 300 may include pre-processing a magnetic resonance image (MRI) of the patient to reconstruct and segment the MRI to form a T1-weighted, T2-weighted, diffusion-weighted, fMRI-weighted, or FLAIR-weighted MRI (step 302). The method 300 may include inputting the T1-weighted, T2-weighted, diffusion-weighted, fMRI-weighted, or FLAIR-weighted MRI into a generative artificial intelligence (“AI”) module 122, the generative AI module 122 configured to compute a training objective for a generative artificial intelligence (“AI”) model based on a training data set of a plurality of cognitively normal brain data sets, each cognitively normal brain data set in the plurality of cognitively normal brain data sets including cognitively normal multi-dimensional brain imaging data corresponding to a cognitively normal brain of a cognitively normal participant (step 304). The method 300 may include receiving, via the generative AI module 122, an estimated saliency map saliency map of the patient for future brain trajectory or future brain disease biomarkers (step 306). The method 300 may include updating, via the generative AI module 122, the generative AI model based on the training objective (step 308).

In various embodiments, in response to the inputting the T1-weighted, T2-weighted, diffusion-weighted, fMRI-weighted, or FLAIR-weighted MRI into the generative AI module 122, the generative AI module 122 determines a saliency map of future brain trajectory or future brain disease biomarkers based on the T1-weighted, T2-weighted, diffusion-weighted, fMRI-weighted, or FLAIR-weighted MRI.

In various embodiments, determining the saliency map of future brain trajectory or future brain disease biomarkers includes calculating an average saliency probability map of future brain trajectory or future brain disease biomarkers based on a sex of the patient.

As used herein, a “script” refers to instructions for a computing device to carry out one or more tasks automatically. As used herein, the term “network” includes any cloud, cloud computing system, or electronic communications system or method which incorporates hardware and/or software components. Communication among the parties may be accomplished through any suitable communication channels, such as, for example, a telephone network, an extranet, an intranet, internet, personal internet device, online communications, satellite communications, off-line communications, wireless communications, transponder communications, local area network (LAN), wide area network (WAN), virtual private network (VPN), networked or linked devices, keyboard, mouse, and/or any suitable communication or data input modality. Moreover, although the system may be described herein as being implemented with TCP/IP communications protocols, the system may also be implemented using IPX, APPLETALK®, IPv6, NetBIOS, any tunneling protocol (e.g. IPsec, SSH, etc.), or any number of existing or future protocols. If the network is in the nature of a public network, such as the internet, it may be advantageous to presume the network to be insecure and open to eavesdroppers. Specific information related to the protocols, standards, and application software utilized in connection with the internet is generally known to those skilled in the art and, as such, need not be detailed herein.

“Cloud” or “Cloud computing” or “cloud computing infrastructure” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand. Reference to a “device” or processor or memory or the like may include cloud resources, non-cloud resources, or combinations of cloud and non-cloud resources.

Computer programs (also referred to as computer control logic) are stored in main memory and/or secondary memory. Computer programs may also be received via communications interface. These computer program instructions may be loaded onto a general purpose computer, special purpose computer, controller, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer, controller, or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

In various embodiments, software may be stored in a computer program product and loaded into a computer system using a removable storage drive, hard disk drive, or communications interface. The control logic (software), when executed by the processor or controller, causes the processor or controller to perform the functions of various embodiments as described herein. In various embodiments, hardware components may take the form of application specific integrated circuits (ASICs). Implementation of the hardware so as to perform the functions described herein will be apparent to persons skilled in the relevant art(s).

As will be appreciated by one of ordinary skill in the art, the system may be embodied as a customization of an existing system, an add-on product, a processing apparatus executing upgraded software, a stand-alone system, a distributed system, a method, a data processing system, a device for data processing, and/or a computer program product. Accordingly, any portion of the system or a module may take the form of a processing apparatus executing code, an internet based embodiment (e.g., an internet-based driving command system), an entirely hardware embodiment, or an embodiment combining aspects of the internet, software, and hardware. Furthermore, the system may take the form of a computer program product on a computer-readable storage medium having computer-readable program code means embodied in the storage medium. Any suitable computer-readable storage medium may be utilized, including hard disks, solid state storage media, CD-ROM, BLU-RAY DISC®, optical storage devices, magnetic storage devices, and/or the like.

The system and method may be described herein in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, JAVA®, JAVASCRIPT®, JAVASCRIPT® Object Notation (JSON), VBScript, Macromedia COLD FUSION, COBOL, MICROSOFT® company's Active Server Pages, assembly, PERL®, PHP, awk, PYTHON®, Visual Basic, SQL Stored Procedures, PL/SQL, any UNIX® shell script, and extensible markup language (XML) with the various algorithms being implemented with any combination of data structures, objects, processes, routines or other programming elements. Further, it should be noted that the system may employ any number of techniques for data transmission, signaling, data processing, network control, and the like. Still further, the system could be used to detect or prevent security issues with a client-side scripting language, such as JAVASCRIPT®, VBScript, or the like.

The system and method are described herein with reference to screen shots, block diagrams and flowchart illustrations of methods, apparatus, and computer program products according to various embodiments. It will be understood that each functional block of the block diagrams and the flowchart illustrations, and combinations of functional blocks in the block diagrams and flowchart illustrations, respectively, can be implemented by computer program instructions.

In various embodiments, components, modules, and/or engines of the systems may be implemented as applications or apps. Apps are typically deployed in the context of a mobile operating system, including for example, a WINDOWS® mobile operating system, an ANDROID® operating system, an APPLE® iOS operating system, a BLACKBERRY® company's operating system, and the like. The app may be configured to leverage the resources of the larger operating system and associated hardware via a set of predetermined rules which govern the operations of various operating systems and hardware resources. For example, where an app desires to communicate with a device or network other than the mobile device or mobile operating system, the app may leverage the communication protocol of the operating system and associated device hardware under the predetermined rules of the mobile operating system. Moreover, where the app desires an input from a user, the app may be configured to request a response from the operating system which monitors various hardware components and then communicates a detected input from the hardware to the app.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows, and the descriptions thereof may make reference to user WINDOWS®/LINUX®/UNIX® applications, webpages, websites, web forms, prompts, etc. Practitioners will appreciate that the illustrated steps described herein may comprise, in any number of configurations, including the use of WINDOWS®/LINUX®/UNIX® applications, webpages, web forms, popup WINDOWS®/LINUX®/UNIX® applications, prompts, and the like. It should be further appreciated that the multiple steps as illustrated and described may be combined into single webpages and/or WINDOWS®/LINUX®/UNIX® applications but have been expanded for the sake of simplicity. In other cases, steps illustrated and described as single process steps may be separated into multiple webpages and/or WINDOWS®/LINUX®/UNIX® applications but have been combined for simplicity.

The computers discussed herein may provide a suitable website or other internet-based graphical user interface (GUI) which is accessible by users. In one embodiment, MICROSOFT® company's Internet Information Services (IIS), Transaction Server (MTS) service, and an SQL SERVER® database, are used in conjunction with MICROSOFT® operating systems, WINDOWS NT® web server software, SQL SERVER® database, and MICROSOFT® Commerce Server. Additionally, components such as ACCESS® software, SQL SERVER® database, ORACLE® software, SYBASE® software, INFORMIX® software, MYSQL® software, INTERBASE® software, etc., may be used to provide an Active Data Object (ADO) compliant database management system. In one embodiment, the APACHE® web server is used in conjunction with a LINUX® operating system, a MYSQL® database, and PHP, Ruby, and/or PYTHON® programming languages.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In re Nuijten to fall outside the scope of patentable subject matter under 35 U.S.C. § 101.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching may be used throughout the figures to denote different parts but not necessarily to denote the same or different materials.

Methods, systems, and articles are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. § 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method, comprising:
computing, via a generative artificial intelligence (“AI”) module, a training objective for a generative artificial intelligence (“AI”) model based on a training data set of a plurality of cognitively normal brain data sets, each cognitively normal brain data set in the plurality of cognitively normal brain data sets including cognitively normal multi-dimensional brain imaging data corresponding to a cognitively normal brain of a cognitively normal participant to form the generative AI model;
receiving, via the generative AI module, a plurality of brain data sets, each of the plurality of brain data sets including subject multi-dimensional brain imaging data, each of the plurality of brain data sets corresponding to a subject brain of each of a plurality of subjects;
extracting, via the generative AI module and from the subject multi-dimensional brain imaging data, a saliency map of future brain trajectory or future brain disease biomarkers for the subject brain of each of the plurality of subjects;
determining, via the generative AI module, a saliency probability map of future brain trajectory or future brain disease biomarkers for each of the plurality of subjects based on the saliency map of future brain trajectory or future brain disease biomarkers and the generative AI model;
estimating, via the generative AI module and through the generative AI model, one of a future brain trajectory or future brain disease biomarkers for the subject brain of each of the plurality of subjects based on the saliency map of future brain trajectory or future brain disease biomarkers; and
updating, via the generative AI module, the generative AI model based on the training objective.

2. The method of claim 1, wherein the training objective of the generative AI model includes a generative neural network or an ensemble of generative, autoencoder, or diffusion neural networks.

3. The method of claim 1, wherein the subject multi-dimensional brain imaging data is a T1-weighted, T2-weighted, diffusion-weighted, fMRI-weighted, or FLAIR-weighted magnetic resonance image.

4. The method of claim 1, wherein determining the saliency probability map of future brain trajectory, including a brain aging trajectory and disease biomarker trajectory, for the subject brain of each of the plurality of subjects includes calculating the saliency probability map of future brain trajectory or future brain disease biomarkers based on a sex of each of the plurality of subjects.

5. The method of claim 4, wherein determining the saliency probability map of future brain trajectory, including the brain aging trajectory and disease biomarker trajectory, for the subject brain of each of the plurality of subjects includes calculating an average of the saliency probability map of future brain trajectory or future brain disease biomarkers based on a deviation from the training data set.

6. The method of claim 1, wherein the saliency probability map of future brain trajectory, including a brain aging trajectory and disease biomarker trajectory, is determined by dividing the saliency probability map of future brain trajectory or future brain disease biomarkers at each of a plurality of brain locations by a sum of all brain saliencies.

7. The method of claim 1, wherein the cognitively normal multi-dimensional brain imaging data is generated by pre-processing a magnetic resonance image for skull-stripping and registering of the cognitively normal brain of the cognitively normal participant into a common coordinate space.

8. The method of claim 1, further comprising:
analyzing, at a surface level, the saliency probability map of future brain trajectory, including brain aging trajectory and disease biomarker trajectory, for the subject brain of each of the plurality of subjects; and
analyzing, at a volume level, the saliency probability map of future brain trajectory, including the brain aging trajectory and disease biomarker trajectory, for the subject brain of each of the plurality of subjects.

9. The method of claim 8, wherein in response to the analyzing at the surface level and the analyzing at the volume level, confounding effects of differences in brain shape and size are removed.

10. The method of claim 8, wherein at the surface level, saliencies from the saliency probability map of future brain trajectory, including the brain aging trajectory and disease biomarker trajectory, are projected to a native cortical surface.

11. The method of claim 8, wherein at the surface level, saliencies from the saliency probability map of future brain trajectory, including the brain aging trajectory and disease biomarker trajectory, are projected onto a cortical mantle as a cortical overlay by volume to surface mapping.

12. The method of claim 8, wherein at the surface level a mean value for the saliency map of future brain trajectory, including the brain aging trajectory and disease biomarker trajectory, is calculated at a surface vertex by averaging the saliency map of future brain trajectory across cortical ribbon voxels within a cylinder according to a Gaussian weighted function.

13. The method of claim 8, wherein the saliency probability map of future brain trajectory, including the brain aging trajectory and disease biomarker trajectory, determined for the subject brain of each of the plurality of subjects is calculated by dividing the saliency probability map of future brain trajectory or future brain disease biomarkers at each of a plurality of brain locations by a sum of all brain saliencies at both the surface level and the volume level.

14. The method of claim 1, further comprising transmitting, via the generative AI module, the saliency probability map of future brain trajectory, including brain aging trajectory and disease biomarker trajectory, for the subject brain of each of the plurality of subjects to a display device.

15. The method of claim 14, wherein the saliency probability map of future brain trajectory, including the brain aging trajectory and disease biomarker trajectory, for the subject brain of each of the plurality of subjects is displayed on the display device in response to the transmitting.

16. A method of estimating a saliency probability map of future brain trajectory, including brain aging trajectory and disease biomarker trajectory, of a patient, the method comprising:
pre-processing a magnetic resonance image (MRI) of the patient to reconstruct and segment the MRI to form a T1-weighted, T2-weighted, diffusion-weighted, fMRI-weighted, or FLAIR-weighted MRI;
inputting the T1-weighted, T2-weighted, diffusion-weighted, fMRI-weighted or FLAIR-weighted MRI into a generative artificial intelligence (“AI”) module, the generative AI module configured to compute a training objective for a generative artificial intelligence (“AI”) model based on a training data set of a plurality of cognitively normal brain data sets, each cognitively normal brain data set in the plurality of cognitively normal brain data sets including cognitively normal multi-dimensional brain imaging data corresponding to a cognitively normal brain of a cognitively normal participant;
receiving, via the generative AI module, an estimated saliency map of the patient for future brain trajectory or future brain disease biomarkers; and
updating, via the generative AI module, the generative AI model based on the training objective.

17. The method of claim 16, wherein in response to the inputting the T1-weighted, T2-weighted, diffusion-weighted, fMRI-weighted, or FLAIR-weighted MRI into the generative AI module, the generative AI module determines a saliency map of future brain trajectory or future brain disease biomarkers based on the T1-weighted, T2-weighted, diffusion-weighted, fMRI-weighted, or FLAIR-weighted MRI.

18. The method of claim 17, wherein determining the saliency map of future brain trajectory or future brain disease biomarkers includes calculating an average saliency probability map of future brain trajectory or future brain disease biomarkers based on a sex of the patient.

19. An article of manufacture including a tangible, non-transitory computer-readable storage medium having instructions stored thereon that, in response to execution by one or more processors, cause the one or more processors to perform operations comprising:
receiving, via the one or more processors, a plurality of brain data sets, each of the plurality of brain data sets including subject multi-dimensional brain imaging data, each of the plurality of brain data sets corresponding to a subject brain of each of a plurality of subjects;
extracting, via the one or more processors, a cognitive or affective saliency map for the subject brain of each of the plurality of subjects;
determining, via the one or more processors, a saliency probability map of future brain trajectory or future brain disease biomarkers for the subject brain of each of the plurality of subjects based on a saliency map of future brain trajectory or future brain disease biomarkers and a generative artificial intelligence (“AI”) model, the generative AI model configured to compute a training objective based on a training data set of a plurality of cognitively normal brain data sets, each cognitively normal brain data set in the plurality of cognitively normal brain data sets including cognitively normal multi-dimensional brain imaging data corresponding to a cognitively normal brain of a cognitively normal participant; and
updating, via the one or more processors, the generative AI model based on the training objective.

20. The article of manufacture of claim 19, wherein the determining the saliency probability map of future brain trajectory, including brain aging trajectory and disease biomarker trajectory, for the subject brain of each of the plurality of subjects includes calculating an average saliency probability map for each sex and a cognitive status.
